(19) United States
(12) Patent Application Publication
(10) Pub. No.: US 2025/0026718 A1
(43) Pub. Date: Jan. 23, 2025

(54) SOLID FORMS OF MESEMBRINE AND THERAPEUTIC USES THEREOF

(71) Applicant: Sensorium Therapeutics, Inc., Boston, MA (US)

(72) Inventor: Zhongyi Lu, Suzhou (CN)

(21) Appl. No.: 18/711,184

(22) PCT Filed: May 23, 2023
(86) PCT No.: PCT/US2023/023215

§ 371 Date: May 17, 2024

(30) Foreign Application Priority Data
May 23, 2022 (WO) PCT/CN2022/094357

(51) Int. Cl. C07D 209/12; A61K 31/404

(52) U.S. Cl. CPC C07D 209/12; A61K 31/404

(57) ABSTRACT

Solid forms of mesembrine are provided, along with related methods of manufacture. The solid form compositions are useful, for example, in the preparation of pharmaceutical compositions.

### RELATED APPLICATIONS

This application claims the benefit of priority to PCT Application No. PCT/CN22/94357, filed May 23, 2022, the contents of which are hereby incorporated by reference in their entirety.

### TECHNICAL FIELD

This disclosure relates to solid forms of mesembrine, and related therapeutic methods of inhibiting the sodium-dependent serotonin transporter (SERT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine and (+)/(−) mesembrenone. The structure of mesembrine, also known as 3a-(3,4-dimethoxyphenyl)-octahydro-1-methyl-6H-indol-6-one, has been reported by Popelak et al., Naturwiss. 47,156 (1960), and the configuration by P W Jeffs et al., J. Am. Chem. Soc. 91, 3831 (1969). Naturally occurring (−) mesembrine (Compound 1) from Sceletium tortuosum has been reported as having serotonin (5-HT) uptake inhibitory activity useful in treating mental health conditions such as mild to moderate depression. Naturally occurring (+)/(−) mesembrenone from Sceletium tortuosum is reported as a potent selective serotonin reuptake inhibitor (Ki=27 nM).

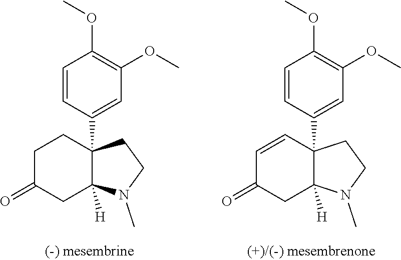 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC]

### Compound 1

Polymorphs, solvates and salts of various drugs have been described in the literature as imparting novel properties to the drugs. Organic small drug molecules have a tendency to self-assemble into various polymorphic forms depending on the environment that drives the self-assembly. Heat and solvent mediated effects can also lead to changes that transform one polymorphic form into another.

Identifying which polymorphic form is the most stable under each condition of interest and the processes that lead to changes in the polymorphic form is crucial to the design of the drug manufacturing process in order to ensure that the final product is in its preferred polymorphic form. Different polymorphic forms of an active pharmaceutical ingredient (API) can lead to changes in the drug's solubility, dissolution rate, pharmacokinetics, and ultimately its bioavailability and efficacy in patients.

### SUMMARY OF THE INVENTION

Described are solid forms of mesembrine (e.g., (−) mesembrine (Compound 1)). In some embodiments, solid forms of mesembrine comprise the product of the processes disclosed herein.

In some embodiments, a pharmaceutical composition comprises a solid form described herein; and a pharmaceutically acceptable excipient.

In some embodiments, a pharmaceutical composition is formed by a process comprising dissolving a solid form described herein.

In some embodiments, a method of treating a mental health disorder, comprises administering to a mammal in need thereof an effective amount of a solid form described herein or a pharmaceutical composition described herein. In some embodiments, the mental health disorder is anxiety, stress, or depression. In some embodiments, the mammal is a human.

### BRIEF DESCRIPTION OF THE DRAWINGS

FIG. 1A is an X-Ray powder diffraction (XRPD) pattern obtained from sample of solid Freeform Form A of Compound 1.

FIG. 1B is a TGA thermogram of material analyzed from room temperature to 350° C. at 10° C. per minute obtained from sample of solid Freeform Form A of Compound 1.

FIG. 1C is a DSC thermogram of material analyzed from room temperature to 230° C. at 10° C. per minute obtained from sample of solid Freeform Form A of Compound 1.

FIG. 1D are XRPD patterns of solid Freeform Form A of Compound 1 before humidity exposure (top), at 25° C./60% RH (middle) for one week, and at 40° C./75% RH (bottom) for one week.

FIG. 1E illustrates the dynamic vapor sorption (DVS) isotherm for solid Freeform Form A of Compound 1, showing desorption and sorption cycling is a DVS isotherm plot obtained from sample of solid Freeform Form A of Compound 1.

FIG. 1F shows X-Ray powder diffraction (XRPD) patterns obtained from a sample of Freeform Form A of Compound 1 showing staring material (top) before and (bottom) after completion of DVS analysis.

FIG. 1G is a ¹H NMR of Compound 1 in DMSO-d6.

FIG. 2A is an X-Ray powder diffraction (XRPD) pattern obtained from sample of solid HCl Form A of Compound 1.

FIG. 2B is a TGA thermogram of material analyzed from room temperature to 350° C. at 10° C. per minute obtained from sample of solid HCl Form A of Compound 1.

FIG. 2C is a DSC thermogram of material analyzed from room temperature to 230° C. at 10° C. per minute obtained from sample of solid HCl Form A of Compound 1.

FIG. 2D is XRPD patterns of solid HCl Form A of Compound 1 before humidity exposure (top), at 25° C./60% RH (middle) for one week, and at 40° C./75% RH (bottom) for one week.

FIG. 2E illustrates the dynamic vapor sorption (DVS) isotherm for solid HCl Form A of Compound 1, showing desorption and sorption cycling is a DVS isotherm plot obtained from sample of solid HCl Form A of Compound 1.

FIG. 2F shows X-Ray powder diffraction (XRPD) patterns obtained from a sample of HCl Form A of Compound 1 showing staring material (top) before and (bottom) after completion of DVS analysis.

FIG. 3A is a X-Ray powder diffraction (XRPD) pattern obtained from sample of solid Tartrate Form A of Compound 1.

FIG. 3B is a TGA thermogram of material analyzed from room temperature to 350° C. at 10° C. per minute obtained from sample of solid Tartrate Form A of Compound 1.

FIG. 3C is a DSC thermogram of material analyzed from room temperature to 230° C. at 10° C. per minute obtained from sample of solid Tartrate Form A of Compound 1.

FIG. 4A is an X-Ray powder diffraction (XRPD) pattern obtained from sample of solid Besylate Form A of Compound 1.

FIG. 4B is a TGA thermogram of material analyzed from room temperature to 350° C. at 10° C. per minute obtained from sample of solid Besylate Form A of Compound 1.

FIG. 4C is a DSC thermogram of material analyzed from room temperature to 230° C. at 10° C. per minute obtained from sample of solid Besylate Form A of Compound 1.

FIG. 5A is an X-Ray powder diffraction (XRPD) pattern obtained from sample of solid Besylate Form B of Compound 1.

FIG. 5B is a TGA thermogram of material analyzed from room temperature to 350° C. at 10° C. per minute obtained from sample of solid Besylate Form B of Compound 1.

FIG. 5C is a DSC thermogram of material analyzed from room temperature to 230° C. at 10° C. per minute obtained from sample of solid Besylate Form B of Compound 1.

FIG. 5D is XRPD patterns of solid Besylate Form B of Compound 1 before humidity exposure (top), at 25° C./60% RH (middle) for one week, and at 40° C./75% RH (bottom) for one week.

FIG. 5E illustrates the dynamic vapor sorption (DVS) isotherm for solid Besylate Form B of Compound 1, showing desorption and sorption cycling is a DVS isotherm plot obtained from sample of solid Besylate Form B of Compound 1.

FIG. 5F shows X-Ray powder diffraction (XRPD) patterns obtained from a sample of Besylate Form B of Compound 1 showing staring material (top) before and (bottom) after completion of DVS analysis.

FIG. 6A is an X-Ray powder diffraction (XRPD) pattern obtained from sample of solid Oxalate Form A of Compound 1.

FIG. 6B is a TGA thermogram of material analyzed from room temperature to 350° C. at 10° C. per minute obtained from sample of solid Oxalate Form A of Compound 1.

FIG. 6C is a DSC thermogram of material analyzed from room temperature to 230° C. at 10° C. per minute obtained from sample of solid Oxalate Form A of Compound 1.

FIG. 7A is an X-Ray powder diffraction (XRPD) pattern obtained from sample of solid Besylate Form B of Compound 1.

FIG. 7B is a TGA thermogram of material analyzed from room temperature to 350° C. at 10° C. per minute obtained from sample of solid Gentisate Form A of Compound 1.

FIG. 7C is a DSC thermogram of material analyzed from room temperature to 230° C. at 10° C. per minute obtained from sample of solid Gentisate Form A of Compound 1.

FIG. 7D is XRPD patterns of solid Gentisate Form A of Compound 1 before humidity exposure (top), at 25° C./60% RH (middle) for one week, and at 40° C./75% RH (bottom) for one week.

FIG. 7E illustrates the dynamic vapor sorption (DVS) isotherm for solid Gentisate Form A of Compound 1, showing desorption and sorption cycling is a DVS isotherm plot obtained from sample of solid Gentisate Form A of Compound 1.

FIG. 7F shows X-Ray powder diffraction (XRPD) patterns obtained from a sample of Gentisate Form A of Compound 1 showing staring material (top) before and (bottom) after completion of DVS analysis.

FIG. 7G shows X-Ray powder diffraction (XRPD) patterns obtained from recovered solids of Gentisate Form A of Compound 1 after being suspended in H₂O for 1 to 24 hours.

FIG. 7H shows X-Ray powder diffraction (XRPD) patterns obtained from recovered solids of Gentisate Form A of Compound 1 after being suspended in SGF for 1 to 24 hours.

FIG. 7I shows X-Ray powder diffraction (XRPD) patterns obtained from recovered solids of Gentisate Form A of Compound 1 after being suspended in FaSSIF for 1 to 24 hours.

FIG. 7J shows X-Ray powder diffraction (XRPD) patterns obtained from recovered solids of Gentisate Form A of Compound 1 after being suspended in FeSSIF for 1 to 24 hours.

FIG. 8A shows the solubility of solid Freeform Form A, solid HCl Form A, solid Besylate Form B, and solid Gentisate Form A in H₂O for 0 to 24 hours.

FIG. 8B shows the solubility of solid Freeform Form A, solid HCl Form A, solid Besylate Form B, and solid Gentisate Form A in SGF for 0 to 24 hours.

FIG. 8C shows the solubility of solid Freeform Form A, solid HCl Form A, solid Besylate Form B, and solid Gentisate Form A in FaSSIF for 0 to 24 hours.

FIG. 8D shows the solubility of solid Freeform Form A, solid HCl Form A, solid Besylate Form B, and solid Gentisate Form A in FeSSIF for 0 to 24 hours.

### DETAILED DESCRIPTION OF THE INVENTION

Applicants have discovered solid forms of mesembrine (e.g., (−) mesembrine (Compound 1)). Although (−) mesembrine is bioactive with certain desirable pharmacologic effects, certain other properties are less than ideal for use as a therapeutic. Solid forms of mesembrine (e.g., crystalline salts of mesembrine) are described herein.

Mesembrine can occur in solid forms as an amorphous solid form or in a crystalline solid form or in mixtures of solid forms. Crystalline solid forms of mesembrine can exist in one or more unique solid forms, which can additionally comprise one or more equivalents of water or solvent (i.e., hydrates or solvates, respectively).

Crystalline form(s) of mesembrine having distinct characteristic XRPD peaks are provided herein. Accordingly, provided herein are crystalline mesembrine solid forms, pharmaceutical compositions thereof, and methods of preparing those crystalline mesembrine solid forms and methods of use thereof.

Compositions comprising the free base of Compound 1 in certain solid forms are provided. Compound 1 can occur in solid forms as an amorphous solid form or in a crystalline solid form or in mixtures of solid forms. Crystalline solid forms of Compound 1 can exist in one or more unique solid forms, which can additionally comprise one or more equivalents of water or solvent (i.e., hydrates or solvates, respectively). Crystalline form(s) of Compound 1 having distinct characteristic XRPD peaks are provided herein. Accordingly, provided herein are crystalline Compound 1 solid forms, pharmaceutical compositions thereof, and methods of preparing those crystalline Compound 1 solid forms and methods of use thereof.

### Free Base Freeform Form A of Compound 1

In some embodiments, anhydrous solid forms of the free base of Compound 1 are provided. Freeform Form A is a solid form of the free base of Compound 1 that can be identified by a XRPD pattern comprising certain characteristic peaks, and/or by one or more other techniques including DSC, TGA, and/or DVS.

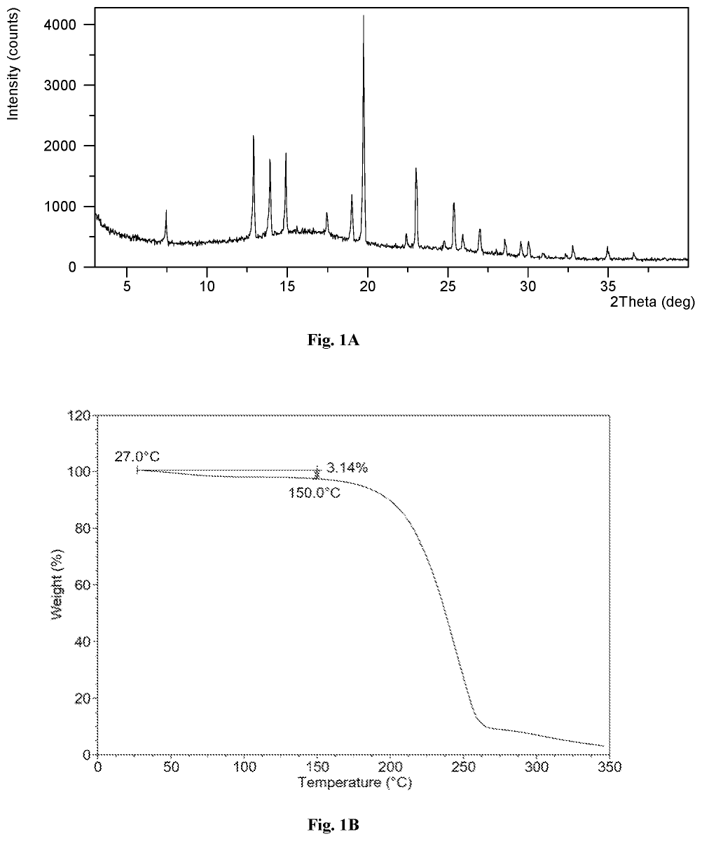

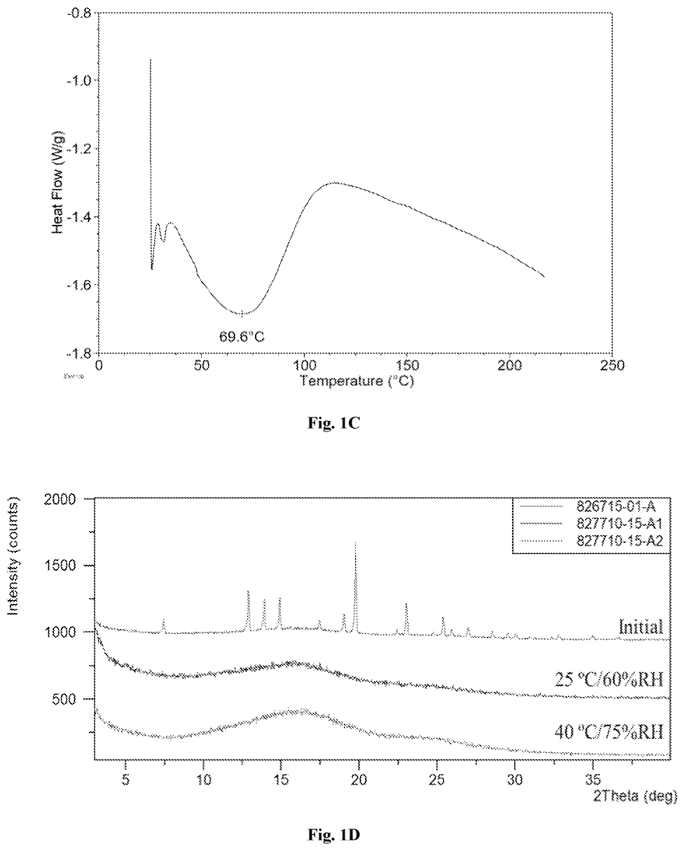

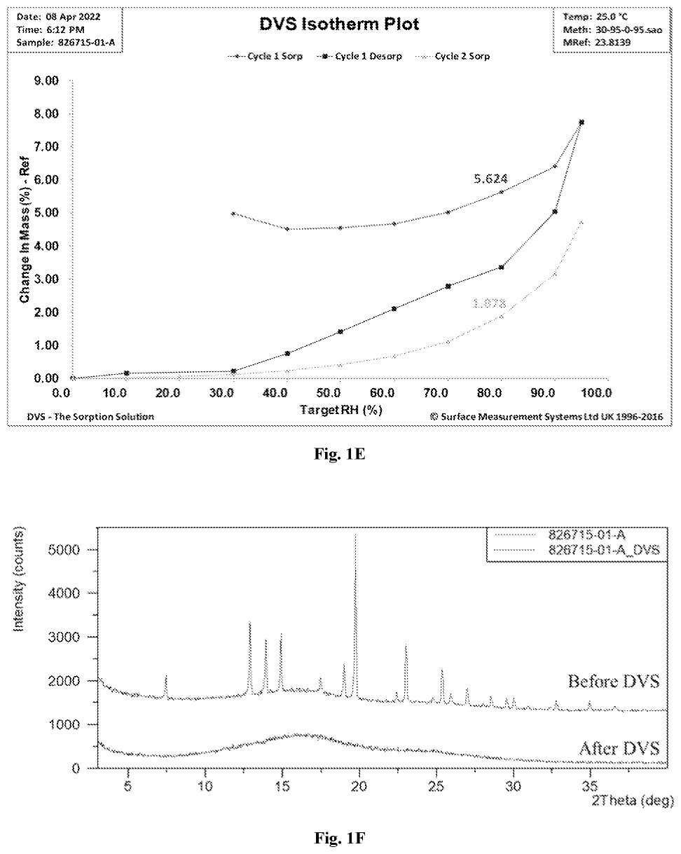

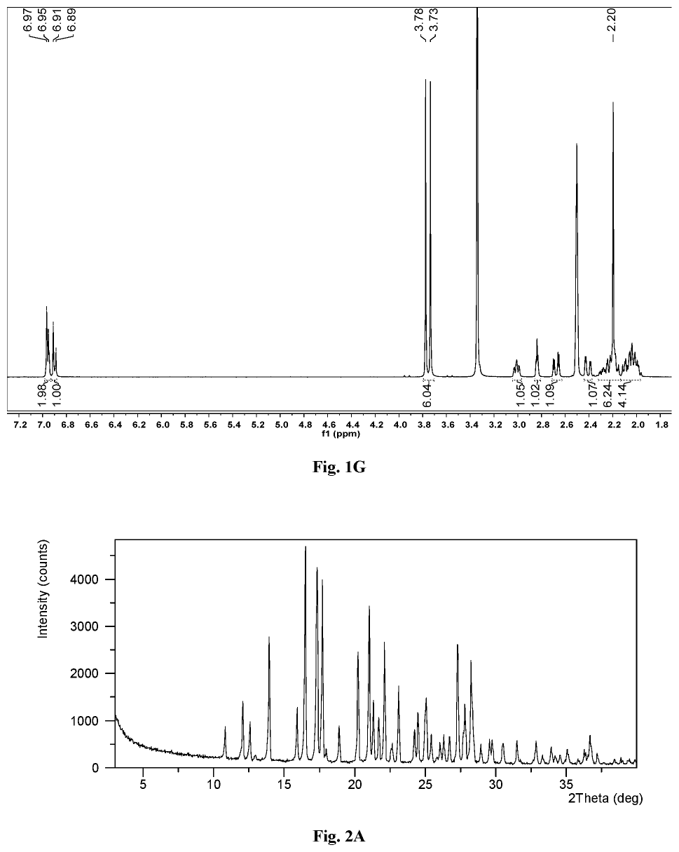

Samples of Freeform Form A were prepared according to the Freeform Example and characterized by XRPD (e.g., a Pattern the same or substantially similar to or not dissimilar to FIG. 1A), TGA (e.g., a thermogram the same or substantially similar to or not dissimilar to FIG. 1B), DSC (e.g., a thermogram the same or substantially similar to or not dissimilar to FIG. 1C), and DVS (e.g., an isotherm the same or substantially similar to or not dissimilar to Figure E).

In some embodiments, Freeform Form A has an X-ray powder diffraction (XRPD) pattern comprising 2-theta peaks (2 theta±0.2) 12.9°, 13.9°, 14.9°, 19.8°, and 23.0°. In some embodiments, Freeform Form A has an X-ray powder diffraction (XRPD) pattern comprising 2-theta peaks (2 theta±0.2) 7.4°, 12.9°, 13.9°, 14.9°, 17.5°, 19.0°, 19.8°, 23.0°, 25.4°, and 27.0°.

In some embodiments, Freeform Form A has an X-ray powder diffraction (XRPD) pattern comprising 2-theta peaks according to Table 4 having a relative intensity of greater than 10%. In some embodiments, Freeform Form A has an X-ray powder diffraction (XRPD) according to FIG. 1A.

In some embodiments, Freeform Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 12.9 | 6.9 |
| 13.9 | 6.4 |
| 14.9 | 5.9 |
| 19.8 | 4.5 |
| 23.0 | 3.9. |

In some embodiments, Freeform Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.4 | 11.9 |
| 12.9 | 6.9 |
| 13.9 | 6.4 |
| 14.9 | 5.9 |
| 17.5 | 5.1 |
| 19.0 | 4.7 |
| 19.8 | 4.5 |
| 23.0 | 3.9 |
| 25.4 | 3.5 |
| 27.0 | 3.3. |

In some embodiments, Freeform Form A is characterized by a TGA thermogram according to FIG. 1B. In some embodiments, Freeform Form A is further characterized by a sample weight loss of up to about 3% upon heating up to 150 C measured by TGA according to the following parameters:

| | |
|---|---|
| Parameters | TGA |
| Method | Ramp |
| Sample pan | Aluminum, open |
| Temperature | RT - 350° C. |
| Heating rate | 10° C./min |
| Purge gas | N₂ |

In some embodiments, Freeform Form A is characterized by a differential scanning calorimetry (DSC) thermogram according to FIG. 1C. In some embodiments, Freeform Form A is characterized by a DSC having a broad endotherm at about 69.6° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

In some embodiments, Freeform Form A is characterized by a DVS isotherm plot according to FIG. 1E.

In some embodiments, Freeform Form A is characterized by an observed solubility of one or more of the following:
a) greater than about 8.3 mg/mL in H₂O;
b) greater than about 8.3 mg/mL in SGF;
c) greater than about 8.3 mg/mL in FaSSIF; or
d) greater than about 8.3 mg/mL in FeSSIF.

### Salt Forms of Compound 1

Various crystalline salts of Compound 1 were prepared, including (but not limited to) crystalline HCl, tartrate, besylate, oxalate, and gentisate forms. In some embodiments, a composition comprises a solid form of mesembrine (e.g., Compound 1 (−) mesembrine) in combination with one or more compounds selected from the group consisting of hydrochloric acid, tartaric acid, oxalic acid, benzensulfonic acid, and gentisic acid.

### HCl Form A

In some embodiments, solid forms of an HCl salt of Compound 1 are provided. HCl Form A is a solid form of the HCl salt of Compound 1 that can be identified by a XRPD pattern comprising certain characteristic peaks, and/or by one or more other techniques including DSC, TGA, and/or DVS.

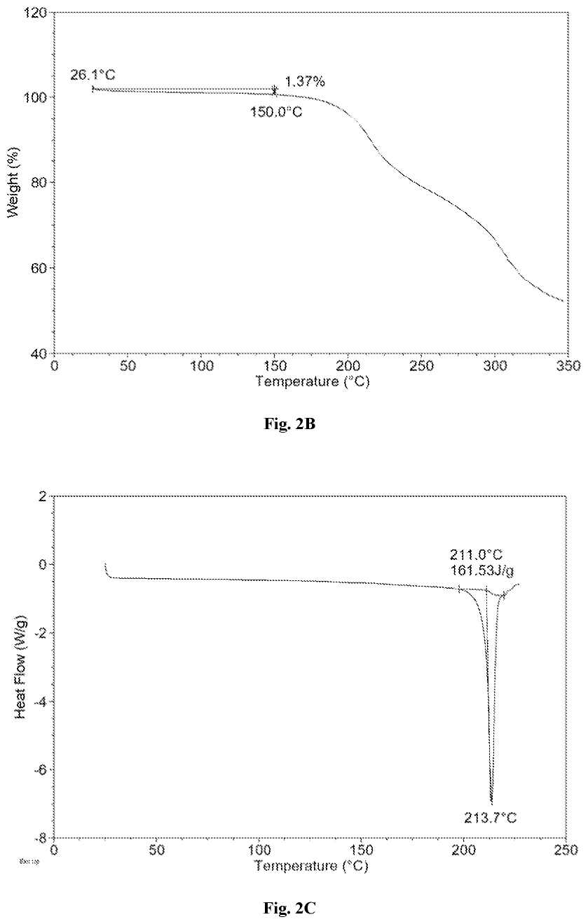

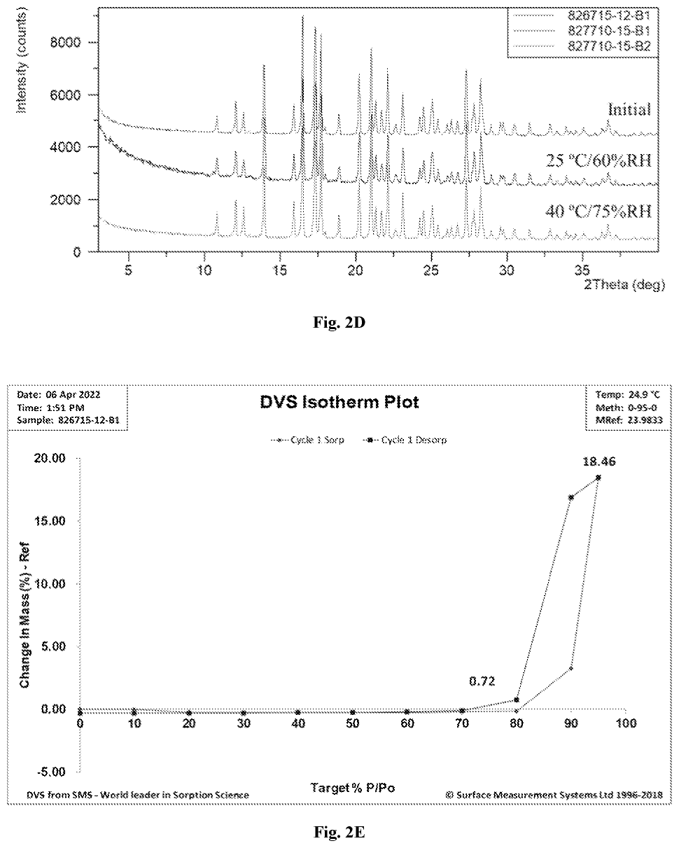

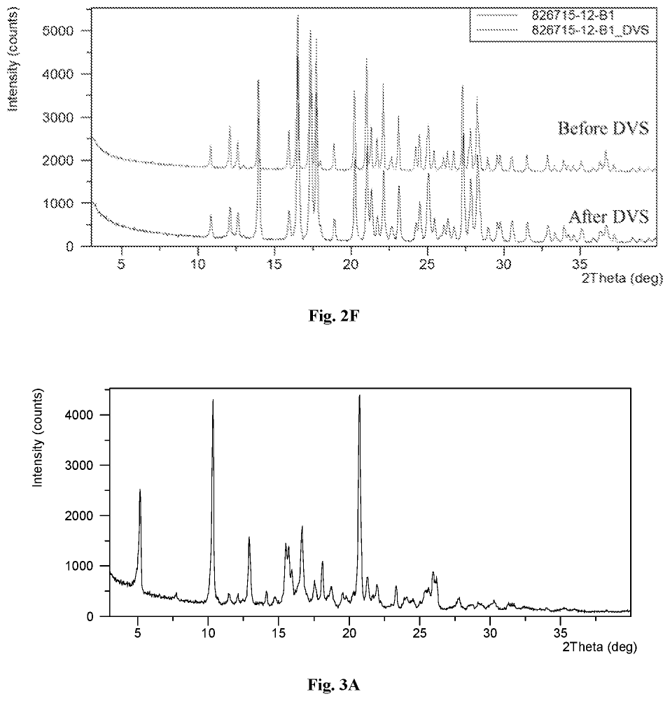

Samples of HCl Form A were prepared according to the Example 1 and characterized by XRPD (e.g., a Pattern the same or substantially similar to or not dissimilar to FIG. 2A), TGA (e.g., a thermogram the same or substantially similar to or not dissimilar to FIG. 2B), DSC (e.g., a thermogram the same or substantially similar to or not dissimilar to FIG. 2C), and DVS (e.g., an isotherm the same or substantially similar to or not dissimilar to FIG. 2E).

In some embodiments, HCl Form A has an X-ray powder diffraction (XRPD) pattern comprising 2-theta peaks (2 theta±0.2) 13.9°, 16.5°, 17.3°, 21.0°, and 27.3°. In some embodiments, HCl Form A has an X-ray powder diffraction (XRPD) pattern comprising 2-theta peaks (2 theta±0.2) 10.8°, 12.1°, 12.6°, 13.9°, 15.9°, 16.5°, 17.3°, 17.7°, 18.9°, 20.2°, 21.0°, 21.3°, 21.7°, 22.1°, 23.1°, 24.2°, 24.5°, 25.1°, 25.4°, 26.3°, 26.7°, 27.3°, 27.8°, 28.2°, 29.5°, 31.5°, and 36.7°.

In some embodiments, HCl Form A has an X-ray powder diffraction (XRPD) pattern comprising 2-theta peaks according to Table 5 having a relative intensity of greater than 10%. In some embodiments, HCl Form A has an X-ray powder diffraction (XRPD) pattern according to FIG. 2A.

In some embodiments, HCl Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 13.9 | 6.4 |
| 16.5 | 5.4 |
| 17.3 | 5.1 |
| 21.0 | 4.2 |
| 27.3 | 3.3 |

In some embodiments, HCl Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 10.8 | 8.2 |
| 12.1 | 7.3 |
| 12.6 | 7.0 |
| 13.9 | 6.4 |
| 15.9 | 5.6 |
| 16.5 | 5.4 |
| 17.4 | 5.1 |
| 17.7 | 5.0 |
| 18.9 | 4.7 |
| 20.2 | 4.4 |
| 21.0 | 4.2 |
| 21.3 | 4.2 |
| 21.7 | 4.1 |
| 22.1 | 4.0 |
| 23.1 | 3.8 |
| 24.2 | 3.7 |
| 24.5 | 3.6 |
| 25.1 | 3.6 |
| 25.4 | 3.5 |
| 26.3 | 3.4 |
| 26.7 | 3.3 |
| 27.3 | 3.3 |
| 27.8 | 3.2 |
| 28.2 | 3.2 |
| 29.5 | 3.0 |
| 31.5 | 2.8 |
| 32.8 | 2.7 |
| 36.7 | 2.4 |

In some embodiments, HCl Form A is characterized by a TGA thermogram according to FIG. 2B. In some embodiments, HCl Form A is further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT - 350° C. |
| Heating rate | 10° C./min |
| Purge gas | N₂ |

In some embodiments, HCl Form A is characterized by a differential scanning calorimetry (DSC) thermogram according to FIG. 2C. In some embodiments, HCl Form A is further characterized by a DSC having an endotherm at about 210° C. to about 215° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

In some embodiments, HCl Form A is characterized by a DVS isotherm plot according to FIG. 2E. In some embodiments, HCl Form A is characterized by substantially the same X-ray powder diffraction (XRPD) pre- and post-DVS according to FIG. 2F. In some embodiments, HCl Form A has a water uptake at 25° C./80% RH of 0.72%, e.g., after one week of storage in the stated condition. No form change was observed by XRPD after DVS test.

In some embodiments, HCl Form A is characterized by an observed solubility of one or more of the following:
a) greater than about 8.3 mg/mL in H₂O;
b) greater than about 8.3 mg/mL in SGF;
c) greater than about 8.3 mg/mL in FaSSIF; or
d) greater than about 8.3 mg/mL in FeSSIF.

In some embodiments, a solid form is produced by a process comprising:
1.) dissolving a free base of (−) mesembrine in acetone to form a (−) mesembrine solution;
2.) adding hydrochloric acid to the (−) mesembrine solution; and
3.) isolating the solids formed in step (2),
   wherein the isolated solids:
a) show a weight loss of about 1% to about 1.5% up to 150° C. by TGA; and/or
b) show an endotherm at about 210° C. to about 215° C. (peak temperature) by DSC.

### Tartrate Form A

In some embodiments, solid forms of a tartrate salt of Compound 1 are provided. Tartrate Form A is a solid form of the Tartrate salt of Compound 1 that can be identified by a XRPD pattern comprising certain characteristic peaks, and/or by one or more other techniques including DSC, TGA, and/or DVS.

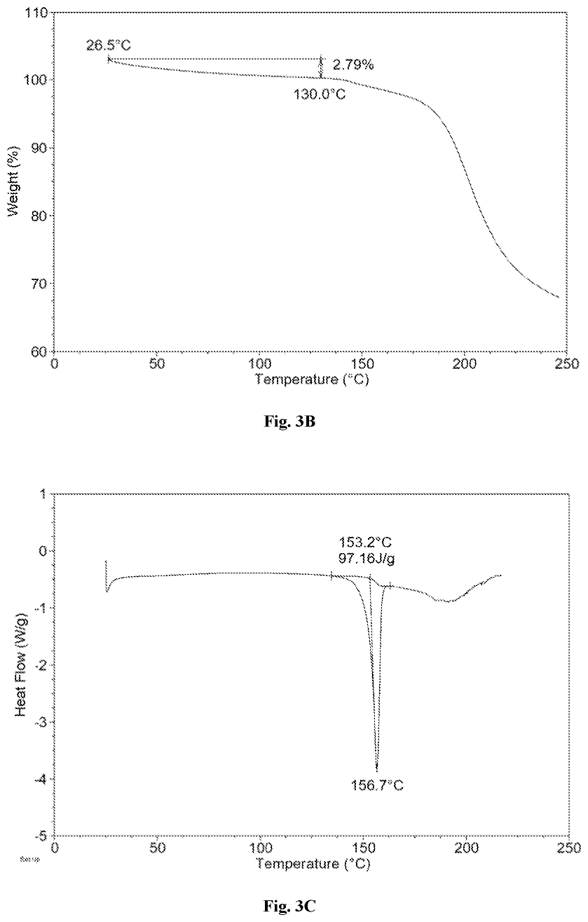

Samples of Tartrate Form A were prepared according to the Example 2 and characterized by XRPD (e.g., a Pattern the same or substantially similar to or not dissimilar to FIG. 3A), TGA (e.g., a thermogram the same or substantially similar to or not dissimilar to FIG. 3B), and DSC (e.g., a thermogram the same or substantially similar to or not dissimilar to FIG. 3C).

In some embodiments, Tartrate Form A has an X-ray powder diffraction (XRPD) pattern comprising 2-theta peaks (2 theta±0.2) 5.2°, 10.3°, 12.9°, 16.7°, and 20.7°. In some embodiments, Tartrate Form A has an X-ray powder diffraction (XRPD) pattern comprising 2-theta peaks (2 theta±0.2) 5.2°, 10.3°, 12.9°, 15.5°, 15.7°, 15.9°, 16.7°, 17.5°, 18.1°, 20.7°, 21.3°, 22.0°, 23.3°, 26.0°, and 26.2°.

In some embodiments, Tartrate Form A has an X-ray powder diffraction (XRPD) pattern comprising 2-theta peaks according to Table 6 having a relative intensity of greater than 10%. In some embodiments, Tartrate Form A has an X-ray powder diffraction (XRPD) pattern according to FIG. 3A.

In some embodiments, Tartrate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 5.2 | 17.1 |
| 10.3 | 8.6 |
| 12.9 | 6.9 |
| 16.7 | 5.3 |
| 20.7 | 4.3 |

In some embodiments, Tartrate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 5.2 | 17.1 |
| 10.3 | 8.6 |
| 12.9 | 6.9 |
| 15.5 | 5.7 |
| 15.7 | 5.6 |
| 15.9 | 5.6 |
| 16.7 | 5.3 |
| 17.5 | 5.1 |
| 18.1 | 4.9 |
| 20.7 | 4.3 |
| 21.3 | 4.2 |
| 22.0 | 4.0 |
| 23.3 | 3.8 |
| 26.0 | 3.4 |
| 26.2 | 3.4 |

In some embodiments, Tartrate Form A is characterized by a TGA thermogram according to FIG. 3B. In some embodiments, Tartrate Form A is further characterized by a sample weight loss of up to about 3% upon heating up to 150° C. measured by TGA according to the following parameters:

| | |
|---|---|
| Parameters | TGA |
| Method | Ramp |
| Sample pan | Aluminum, open |
| Temperature | RT - 350° C. |
| Heating rate | 10° C./min |
| Purge gas | N₂ |

In some embodiments, Tartrate Form A is characterized by a differential scanning calorimetry (DSC) thermogram according to FIG. 3C. In some embodiments, Tartrate Form A is further characterized by a DSC having an endotherm at about 155° C. to about 160° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

In some embodiments, a solid form is produced by a process of:
1.) dissolving a free base of (−) mesembrine in ethanol to form a (−) mesembrine solution;
2.) adding 1-tartaric acid to the (−) mesembrine solution; and
3.) isolating the solids formed in step (2),
   wherein the isolated solids:
a) show a weight loss of about 2% to about 3% up to 150° C. by TGA; and/or
b) show an endotherm at about 155° C. to about 160° C. (peak temperature) by DSC.

### Besylate Form A

In some embodiments, solid forms of a besylate salt of Compound 1 are provided. Besylate Form A is a solid form of the Besylate salt of Compound 1 that can be identified by a XRPD pattern comprising certain characteristic peaks, and/or by one or more other techniques including DSC, TGA, and/or DVS.

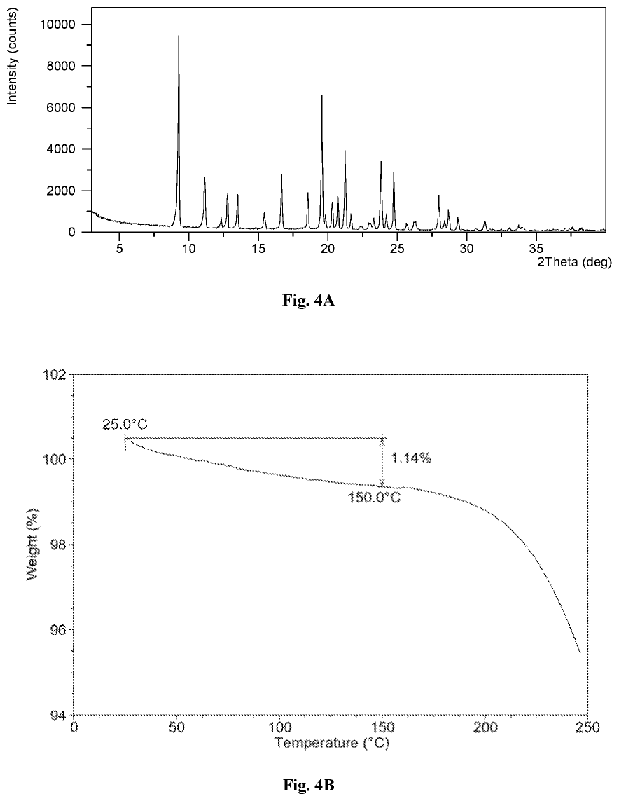

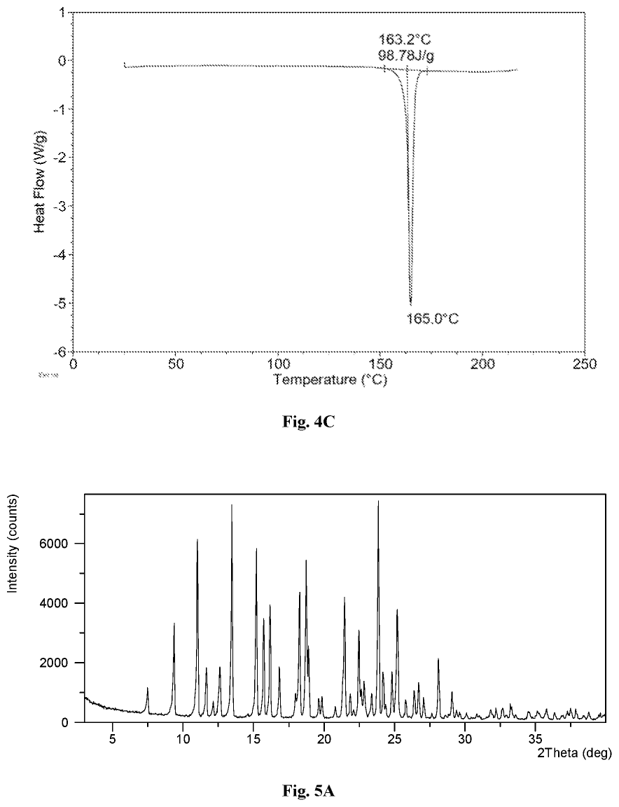

Samples of Besylate Form A were prepared according to the Example 3 and characterized by XRPD (e.g., a Pattern the same or substantially similar to or not dissimilar to FIG. 4A), TGA (e.g., a thermogram the same or substantially similar to or not dissimilar to FIG. 4B), and DSC (e.g., a thermogram the same or substantially similar to or not dissimilar to FIG. 4C).

In some embodiments, Besylate Form A has an X-ray powder diffraction (XRPD) comprising 2-theta peaks (2 theta±0.2) 9.3°, 19.6°, 21.2°, 23.8°, and 24.7°. In some embodiments, Besylate Form A has an X-ray powder diffraction (XRPD) pattern comprising 2-theta peaks (2 theta±0.2) 9.3°, 11.1°, 12.8°, 13.5°, 16.7°, 18.6°, 19.6°, 20.3°, 20.7°, 21.2°, 23.8°, 24.7°, and 28.0°.

In some embodiments, Besylate Form A has an X-ray powder diffraction (XRPD) pattern comprising 2-theta peaks according to Table 7 having a relative intensity of greater than 10%. In some embodiments, Besylate Form A has an X-ray powder diffraction (XRPD) pattern according to FIG. 4A.

In some embodiments, Besylate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.3 | 9.5 |
| 19.6 | 4.5 |
| 21.2 | 4.2 |
| 23.8 | 3.7 |
| 24.7 | 3.6 |

In some embodiments, Besylate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.3 | 9.5 |
| 11.1 | 7.9 |
| 12.8 | 6.9 |
| 13.5 | 6.6 |
| 16.7 | 5.3 |
| 18.6 | 4.8 |
| 19.6 | 4.5 |
| 20.3 | 4.4 |
| 20.7 | 4.3 |
| 21.2 | 4.2 |
| 23.8 | 3.7 |
| 24.7 | 3.6 |
| 28.0 | 3.2 |

In some embodiments, Besylate Form A is characterized by a TGA thermogram according to FIG. 4B. In some embodiments, Besylate Form A is further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT- 350° C. |
| Heating rate | 10° C./min |
| Purge gas | N₂ |

In some embodiments, Besylate Form A is characterized by a differential scanning calorimetry (DSC) thermogram according to FIG. 4C. In some embodiments, Besylate Form A is further characterized by a DSC having an endotherm at about 163° C. to about 168° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

In some embodiments, a solid form produced by a process of:
1.) dissolving a free base of (−) mesembrine in acetone to form a (−) mesembrine solution;
2.) adding benzenesulfonic acid to the (−) mesembrine solution; and
3.) isolating the solids formed in step (2),
   wherein the isolated solids:
a) show a weight loss of about 1% to about 1.5% up to 150° C. by TGA; and/or
b) show an endotherm at about 163° C. to about 168° C. (peak temperature) by DSC.

### Besylate Form B

In some embodiments, solid forms of a besylate salt of Compound 1 are provided. Besylate Form B is a solid form of the Besylate salt of Compound 1 that can be identified by a XRPD pattern comprising certain characteristic peaks, and/or by one or more other techniques including DSC, TGA, and/or DVS.

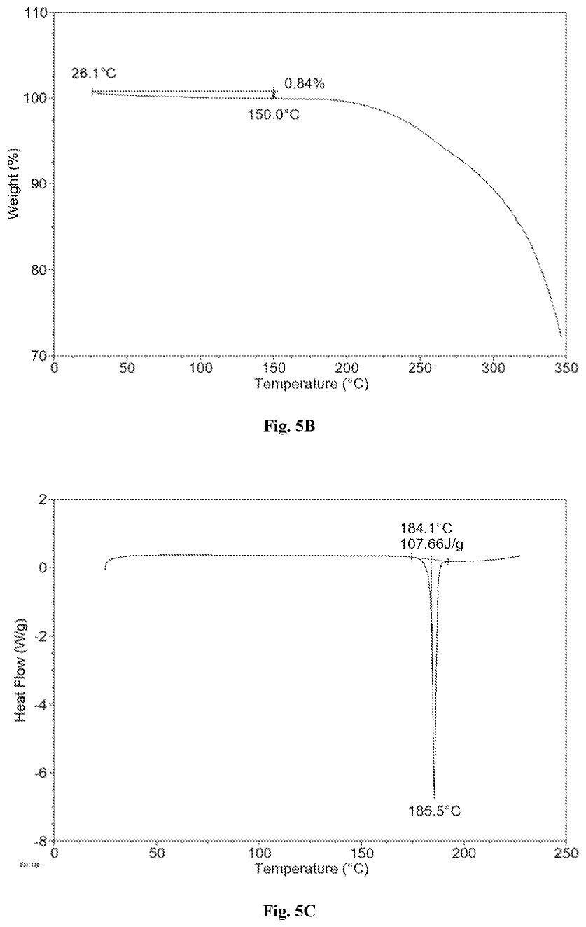

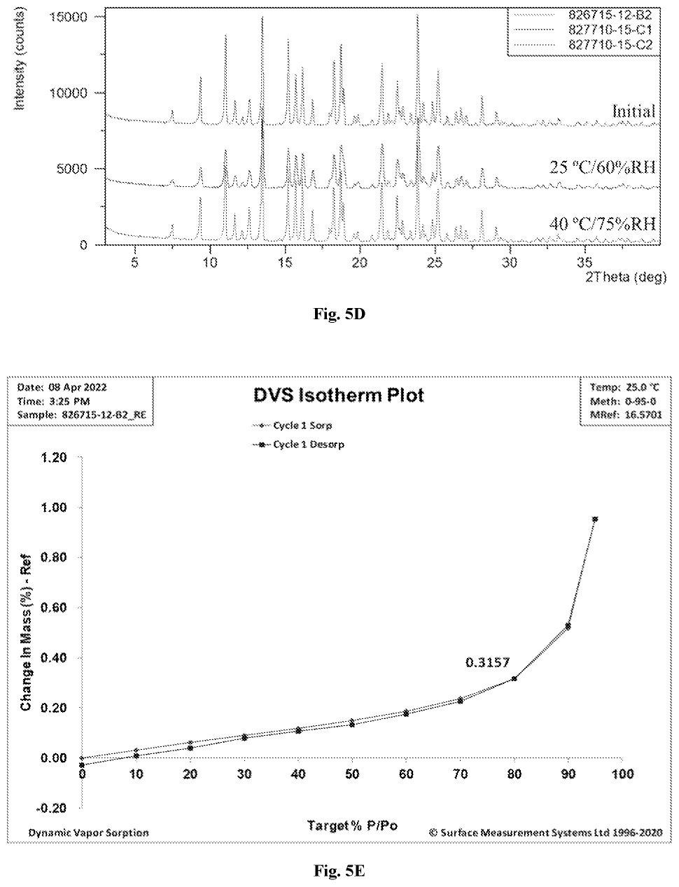

Samples of Besylate Form B were prepared according to the Example 4 and characterized by XRPD (e.g., a Pattern the same or substantially similar to or not dissimilar to FIG. 5A), TGA (e.g., a thermogram the same or substantially similar to or not dissimilar to FIG. 5B), DSC (e.g., a thermogram the same or substantially similar to or not dissimilar to FIG. 5C), and DVS (e.g., an isotherm the same or substantially similar to or not dissimilar to FIG. 5E).

In some embodiments, Besylate Form B has an X-ray powder diffraction (XRPD) comprising an X-ray powder diffraction (XRPD) pattern comprising 2-theta peaks (2 theta±0.2) 11.0°, 13.5°, 15.2°, 18.7°, and 23.8°. In some embodiments, Besylate Form B has an an X-ray powder diffraction (XRPD) pattern comprising 2-theta peaks (2 theta±0.2) 7.5°, 9.4°, 11.0°, 11.7°, 12.6°, 13.5°, 15.2°, 15.7°, 16.2°, 16.8°, 18.0°, 18.3°, 18.7°, 18.9°, 21.5°, 21.9°, 22.5°, 22.8°, 23.4°, 23.8°, 24.2°, 24.8°, 25.2°, 26.4°, 26.7°, 28.1°, and 29.1°.

In some embodiments, Besylate Form B has an X-ray powder diffraction (XRPD) pattern comprising 2-theta peaks according to Table 8 having a relative intensity of greater than 10%. In some embodiments, Besylate Form B has an X-ray powder diffraction (XRPD) pattern according to FIG. 5A.

In some embodiments, Besylate Form B is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 11.0 | 8.0 |
| 13.5 | 6.6 |
| 15.2 | 5.8 |
| 18.7 | 4.7 |
| 23.8 | 3.7 |

In some embodiments, Besylate Form B is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.5 | 11.8 |
| 9.4 | 9.4 |
| 11.0 | 8.0 |
| 11.7 | 7.6 |
| 12.6 | 7.0 |
| 13.5 | 6.6 |
| 15.2 | 5.8 |
| 15.7 | 5.6 |
| 16.2 | 5.5 |
| 16.8 | 5.3 |
| 18.0 | 4.9 |
| 18.3 | 4.9 |
| 18.7 | 4.7 |
| 18.9 | 4.7 |
| 21.5 | 4.1 |
| 21.9 | 4.1 |
| 22.5 | 4.0 |
| 22.8 | 3.9 |
| 23.4 | 3.8 |
| 23.9 | 3.7 |
| 24.2 | 3.7 |
| 24.8 | 3.6 |
| 25.2 | 3.5 |
| 26.4 | 3.4 |
| 26.7 | 3.3 |
| 28.1 | 3.2 |
| 29.1 | 3.1. |

In some embodiments, Besylate Form B is characterized by a TGA thermogram according to FIG. 5B. In some embodiments, Besylate Form B is further characterized by a sample weight loss of up to about 1% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

In some embodiments, Besylate Form B is characterized by a differential scanning calorimetry (DSC) thermogram according to FIG. 5C. In some embodiments, Besylate Form B is characterized by a DSC having endotherm at about 183° C. to about 188° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

In some embodiments, Besylate Form B is characterized by substantially the same X-ray powder diffraction (XRPD) pattern of the bottom panel of FIG. 5D post storage at 40° C. and 75% RH for at least a week.

In some embodiments, Besylate Form B is characterized by a DVS isotherm plot according to FIG. 5E. In some embodiments, Besylate Form B is characterized by substantially the same X-ray powder diffraction (XRPD) pre- and post-DVS according to FIG. 5F. No form change was observed by XRPD after DVS test.

In some embodiments, Besylate Form B is has a water uptake at 25° C./80% RH of 0.32%, e.g., after one week of storage at the recited conditions.

In some embodiments, Besylate Form B is characterized by an observed solubility of one or more of the following:
a) greater than about 8.3 mg/mL in H₂O;
b) greater than about 8.3 mg/mL in SGF;
c) greater than about 8.3 mg/mL in FaSSIF; or
d) greater than about 8.3 mg/mL in FeSSIF.

In some embodiments, a solid form is produced by a process of:
1.) combining a free base of (−) mesembrine and benzenesulfonic acid;
2.) adding acetone to the mixture of step (1); and
3.) isolating the solids formed in step (2),
   wherein the isolated solids:
a) show a weight loss of about 0.5% to about 1% up to 150° C. by TGA; and/or
b) show an endotherm at about 183° C. to about 188° C. (peak temperature) by DSC.

### Oxalate Form A

In some embodiments, solid forms of an oxalate salt of Compound 1 are provided. Oxalate Form A is a solid form of the oxalate salt of Compound 1 that can be identified by a XRPD pattern comprising certain characteristic peaks, and/or by one or more other techniques including DSC, TGA, and/or DVS.

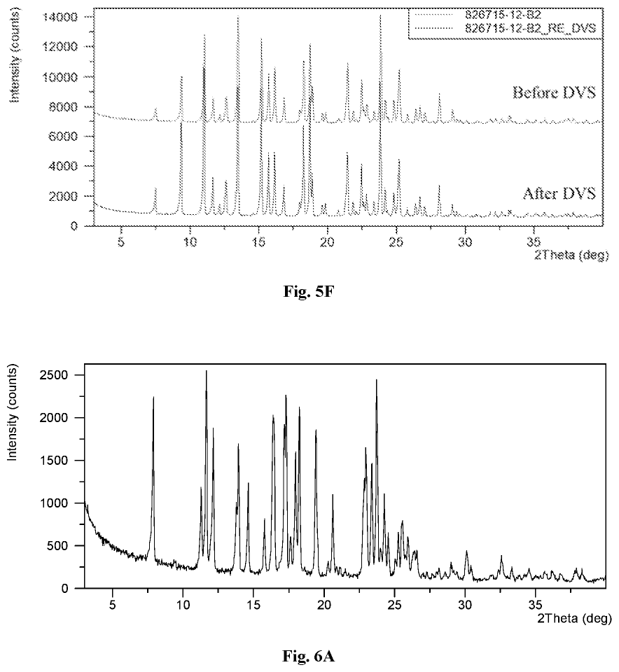

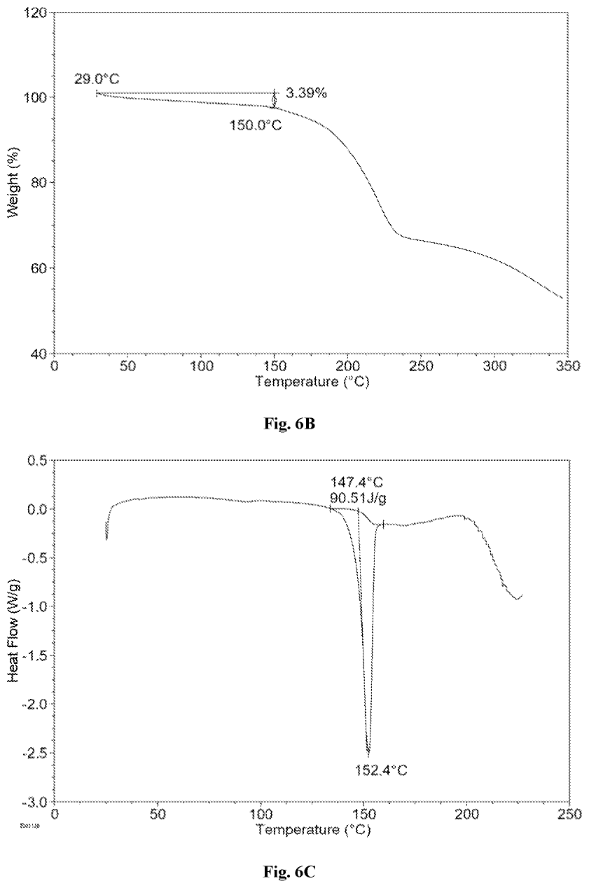

Samples of Oxalate Form A were prepared according to the Example 5 and characterized by XRPD (e.g., a Pattern the same or substantially similar to or not dissimilar to FIG. 6A), TGA (e.g., a thermogram the same or substantially similar to or not dissimilar to FIG. 6B), and DSC (e.g., a thermogram the same or substantially similar to or not dissimilar to FIG. 6C).

In some embodiments, Oxalate Form A has an X-ray powder diffraction (XRPD) comprising an X-ray powder diffraction (XRPD) pattern comprising 2-theta peaks (2 theta±0.2) 7.9°, 11.7°, 17.3°, 18.2°, and 23.7°. In some embodiments, Oxalate Form A has an X-ray powder diffraction (XRPD) pattern comprising 2-theta peaks (2 theta±0.2) 7.9°, 11.3°, 11.7°, 12.2°, 13.8°, 13.9°, 14.6°, 15.8°, 16.5, 17.2°, 17.3°, 17.6°, 18.0°, 18.2°, 19.4°, 20.6°, 22.8°, 23.0°, 23.4°, 23.7°, 24.0°, 24.3°, 24.6°, 25.3°, 25.5°, 26.0°, 26.4°, 26.6°, 30.1°, and 32.6°.

In some embodiments, Oxalate Form A has an X-ray powder diffraction (XRPD) pattern comprising 2-theta peaks according to Table 9 having a relative intensity of greater than 10%. In some embodiments, Oxalate Form A has an X-ray powder diffraction (XRPD) pattern according to FIG. 6A.

In some embodiments, Oxalate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.9 | 11.2 |
| 11.7 | 7.6 |
| 17.3 | 5.1 |
| 18.2 | 4.9 |
| 23.7 | 3.8. |

In some embodiments, Oxalate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.9 | 11.2 |
| 11.3 | 7.8 |
| 11.7 | 7.6 |
| 12.2 | 7.3 |
| 13.8 | 6.4 |
| 13.9 | 6.4 |
| 14.6 | 6.1 |
| 15.8 | 5.6 |
| 16.5 | 5.4 |
| 17.2 | 5.2 |
| 17.3 | 5.1 |
| 17.6 | 5.0 |
| 18.0 | 4.9 |
| 18.2 | 4.9 |
| 19.4 | 4.6 |
| 20.6 | 4.3 |
| 22.8 | 3.9 |
| 23.0 | 3.9 |
| 23.4 | 3.8 |
| 23.7 | 3.8 |
| 24.0 | 3.7 |
| 24.3 | 3.7 |
| 24.6 | 3.6 |
| 25.3 | 3.5 |
| 25.5 | 3.5 |
| 26.0 | 3.4 |
| 26.4 | 3.4 |
| 26.6 | 3.4 |
| 30.1 | 3.0 |
| 32.6 | 2.8. |

In some embodiments, Oxalate Form A is characterized by a TGA thermogram according to FIG. 6B. In some embodiments, Oxalate Form A is further characterized by a sample weight loss of up to about 3.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

In some embodiments, Oxalate Form A is characterized by a differential scanning calorimetry (DSC) thermogram according to FIG. 6C. In some embodiments, Oxalate Form A is further characterized by a DSC having endotherm at about 150° C. to about 155° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

In some embodiments, a solid form is produced by a process of:
1.) dissolving a free base of (−) mesembrine in acetone to form a (−) mesembrine solution;
2.) adding oxalic acid to the (−) mesembrine solution; and
3.) isolating the solids formed in step (2),
   wherein the isolated solids:
a) show a weight loss of about 3% to about 3.5% up to 150° C. by TGA; and/or
b) show an endotherm at about 150° C. to about 155° C. (peak temperature) by DSC.

### Gentisate Form A

In some embodiments, solid forms of a gentisate salt of Compound 1 are provided. Gentisate Form A is a solid form of the gentisate salt of Compound 1 that can be identified by a XRPD pattern comprising certain characteristic peaks, and/or by one or more other techniques including DSC, TGA, and/or DVS.

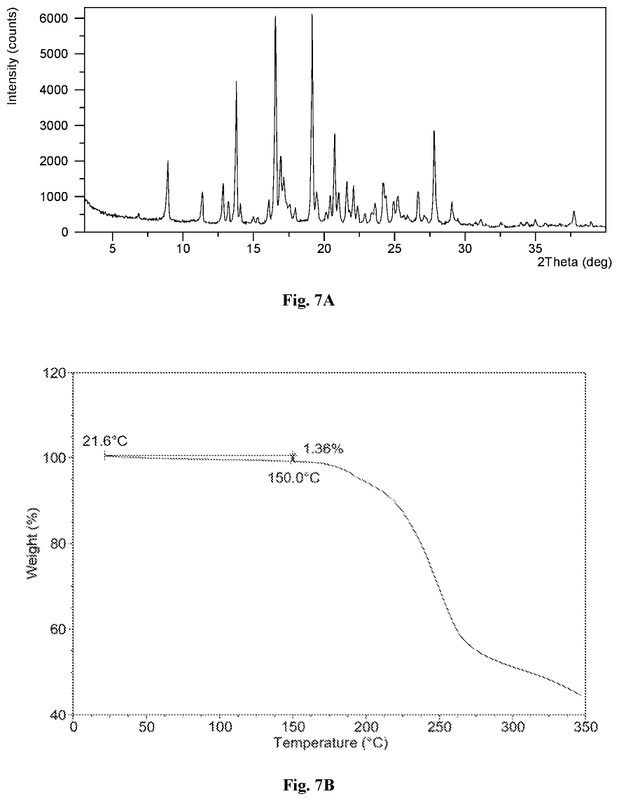

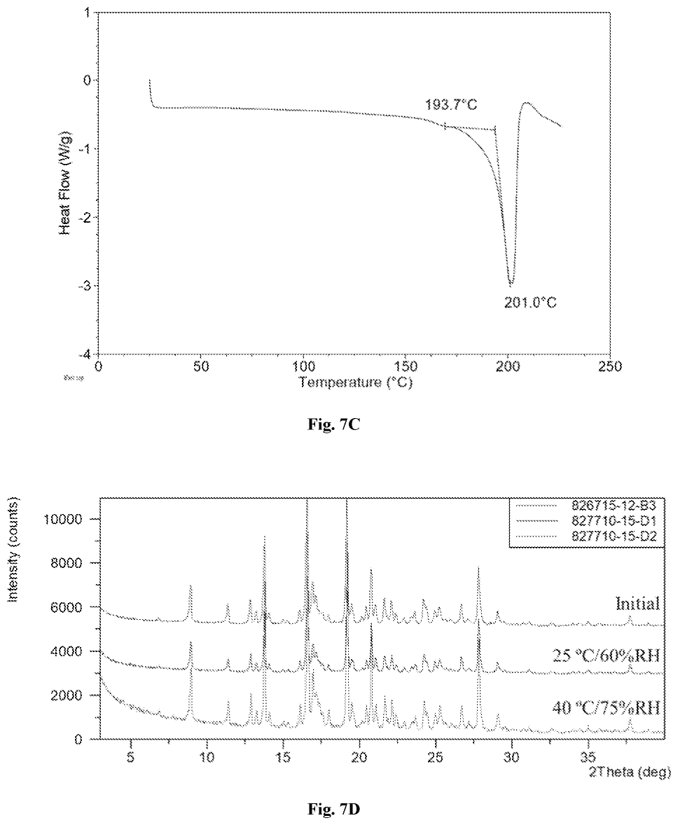

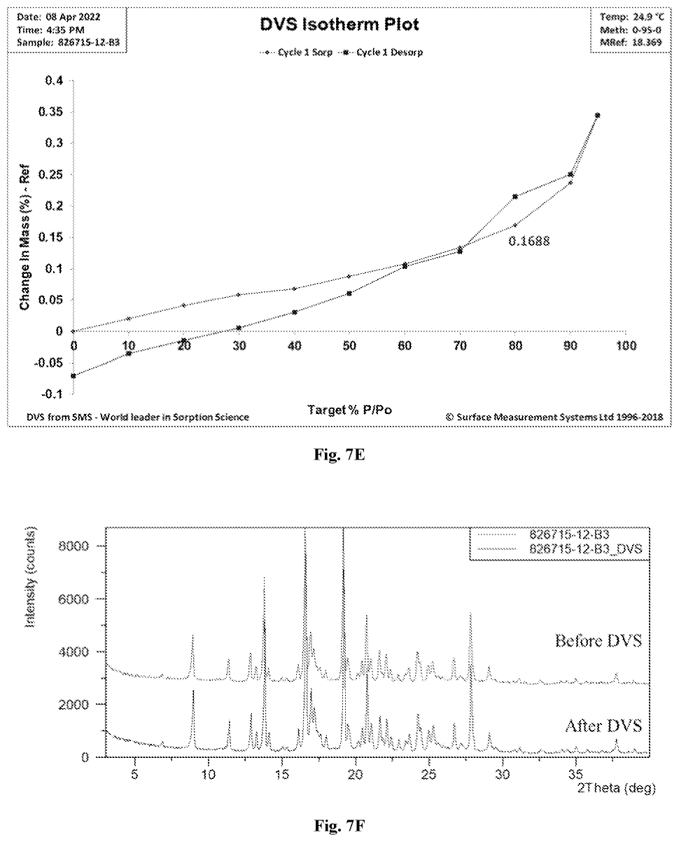

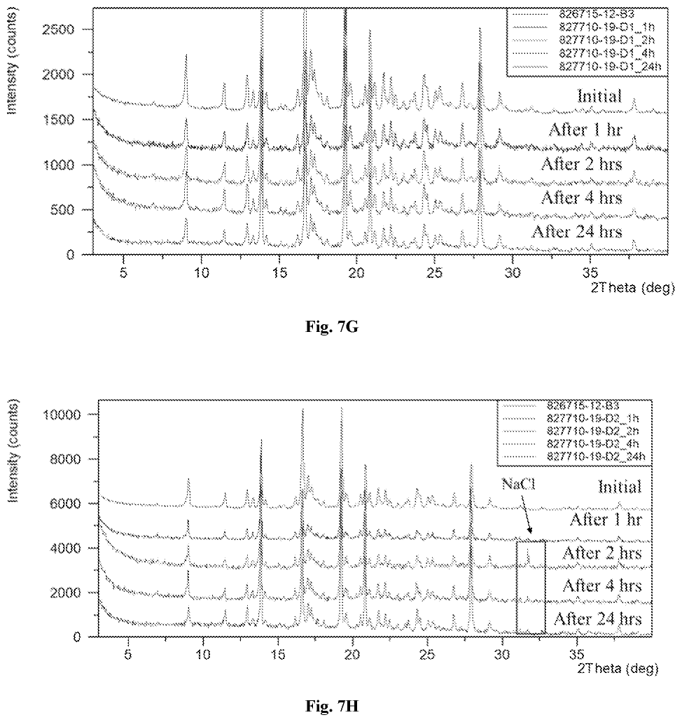

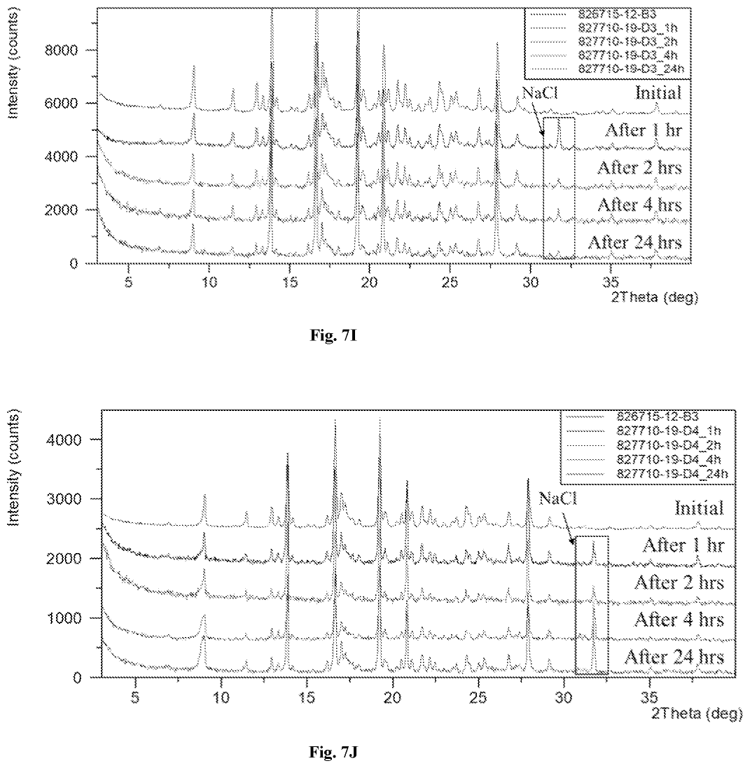

Samples of Gentisate Form A were prepared according to the Example 6 and characterized by XRPD (e.g., a Pattern the same or substantially similar to or not dissimilar to FIG. 7A), TGA (e.g., a thermogram the same or substantially similar to or not dissimilar to FIG. 7B), DSC (e.g., a thermogram the same or substantially similar to or not dissimilar to FIG. 7C), and DVS (e.g., an isotherm the same or substantially similar to or not dissimilar to FIG. 7E).

In some embodiments, Gentisate Form A has an X-ray powder diffraction (XRPD) pattern comprising 2-theta peaks (2 theta±0.2) 13.8°, 16.6°, 19.2°, 20.8°, and 27.8°. In some embodiments, Gentisate Form A has an X-ray powder diffraction (XRPD) pattern comprising 2-theta peaks (2 theta±0.2) 8.9°, 11.4°, 12.8°, 13.2°, 13.8°, 16.1°, 16.6°, 16.9°, 17.2°, 19.2°, 19.5°, 20.4°, 20.8°, 21.0°, 21.6°, 22.1°, 24.2°, 24.4°, 24.9°, 25.2°, 26.7°, 27.8°, and 29.1°.

In some embodiments, Gentisate Form A has an X-ray powder diffraction (XRPD) pattern comprising 2-theta peaks according to Table 10 having a relative intensity of greater than 10%. In some embodiments, Gentisate Form A has an X-ray powder diffraction (XRPD) pattern according to FIG. 7A.

In some embodiments, Gentisate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 13.8 | 6.4 |
| 16.6 | 5.4 |
| 19.2 | 4.6 |
| 20.8 | 4.3 |
| 27.8 | 3.2. |

In some embodiments, Gentisate Form A is characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 8.9 | 9.9 |
| 11.4 | 7.8 |
| 12.8 | 6.9 |
| 13.2 | 6.7 |
| 13.8 | 6.4 |
| 16.1 | 5.5 |
| 16.6 | 5.4 |
| 16.9 | 5.2 |
| 17.2 | 5.2 |
| 19.2 | 4.6 |
| 19.5 | 4.6 |
| 20.4 | 4.4 |
| 20.8 | 4.3 |
| 21.0 | 4.2 |
| 21.6 | 4.1 |
| 22.1 | 4.0 |
| 24.2 | 3.7 |
| 24.4 | 3.6 |
| 24.9 | 3.6 |
| 25.2 | 3.5 |
| 26.7 | 3.3 |
| 27.8 | 3.2 |
| 29.1 | 3.1. |

In some embodiments, Gentisate Form A is characterized by a TGA thermogram according to FIG. 7B. In some embodiments, Gentisate Form A is further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

In some embodiments, Gentisate Form A is characterized by a differential scanning calorimetry (DSC) thermogram according to FIG. 7C. In some embodiments, Gentisate Form A is further characterized by a DSC having an endotherm at about 198° C. to about 203° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

In some embodiments, Gentisate Form A is characterized by substantially the same X-ray powder diffraction (XRPD) pattern of the bottom panel of FIG. 7D post storage at 40° C. and 75% RH for at least a week.

In some embodiments, Gentisate Form A is characterized by a DVS isotherm plot according to FIG. 7E. In some embodiments, Gentisate Form A is characterized by substantially the same X-ray powder diffraction (XRPD) pre- and post-DVS according to FIG. 7F. No form change was observed by XRPD after DVS test.

In some embodiments, Gentisate Form A has a water uptake at 25° C./80% RH of 0.17%, e.g., after one week in the stated conditions.

In some embodiments, Gentisate Form A is characterized by an observed solubility of one or more of the following:
a) greater than about 3 mg/mL in H₂O;
b) greater than about 5.5 mg/mL in SGF;
c) greater than about 3.5 mg/mL in FaSSIF; or
d) greater than about 4 mg/mL in FeSSIF.

In some embodiments, a solid form is produced by a process of:
1.) combining a free base of (−) mesembrine and gentisic acid;
2.) adding ethanol to the mixture of step (1); and
3.) isolating the solids formed in step (2),
   wherein the isolated solids:
a) show a weight loss of about 1% to about 1.5% up to 150° C. by TGA; and/or
b) show an endotherm at about 198° C. to about 203° C. (peak temperature) by DSC.

### Pharmaceutical Compositions & Methods of Treatment

In some embodiments, pharmaceutical compositions comprising mesembrine, and pharmaceutically acceptable salts and hydrates thereof are provided. The pharmaceutical composition can comprise mesembrine in one or more solid forms provided herein, such as crystalline mesembrine in a hydrated or anhydrous solid form. A pharmaceutical composition, as used herein, refers to a mixture of mesembrine optionally further comprising other pharmaceutically acceptable components, such as carriers, stabilizers, diluents, dispersing agents, suspending agents, thickening agents, and/or excipients. The pharmaceutical composition can facilitate administration of the compound to a mammal, including compositions formulated for oral administration of mesembrine to a mammal (e.g., capsules or tablets).

In some embodiments, crystalline mesembrine is incorporated into pharmaceutical compositions to provide solid oral dosage forms. In other embodiments, crystalline mesembrine is used to prepare pharmaceutical compositions prepared for oral solid dosage forms. In some embodiments, the pharmaceutical composition comprises an active pharmaceutical ingredient (API) comprising, consisting essentially of, or consisting of mesembrine prepared under applicable Good Manufacturing Practice (GMP). For example, the pharmaceutical composition can be a batch composition comprising mesembrine, wherein the batch composition adheres to Good Manufacturing Practices (e.g., ICH Harmonised Tripartite Guideline, Good Manufacturing Practice Guide for Active Pharmaceutical Ingredients Q7, Current Step 4 version dated 10 Nov. 2010). More preferably, the GMP batch composition can be a homogenous blended batch comprising mesembrine. The FDA (Food and Drug Administration) provides applicable guidance on Good Manufacturing Practice (GMP) for the manufacturing of active pharmaceutical ingredients (APIs) under an appropriate system for managing quality. As used with respect to manufacture of API under GMP, “manufacturing” is defined to include all operations of receipt of materials, production, packaging, repackaging, labelling, relabeling, quality control, release, storage and distribution of APIs and the related controls. An “API Starting Material” is a raw material, intermediate, or an API that is used in the production of an API and that is incorporated as a significant structural fragment into the structure of the API. An API Starting Material can be an article of commerce, a material purchased from one or more suppliers under contract or commercial agreement, or produced in-house. API Starting Materials normally have defined chemical properties and structure.

The pharmaceutical compositions comprising mesembrine can be administered to patients in need thereof, to provide a therapeutically effective amount of a compound of mesembrine.

In practicing the methods of treatment or use provided herein, therapeutically effective amounts of mesembrine are administered in a pharmaceutical composition to a mammal having a disease, disorder, or condition to be treated. In some embodiments, the disease, disorder, or condition is a central nervous system disorder or an inflammatory condition. In some embodiments, pharmaceutical compositions reported herein can be provided in a unit dosage form container (e.g., in a vial or bag or the like).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a mesembrine composition for the treatment of a disease selected from the group consisting of mild to moderate depression and major depressive episodes. In some embodiments, a method comprises the administration to a patient in need thereof of a therapeutically effective amount of a mesembrine composition for the treatment of anxiety. In some embodiments, a method comprises the administration to a patient in need thereof of a therapeutically effective amount of a mesembrine composition for the treatment of depression. In some embodiments, a method comprises the administration to a patient in need thereof of a therapeutically effective amount of a mesembrine composition for the treatment of a condition selected from the group consisting of: anxiety associated with depression, anxiety with depression, mixed anxiety and depressive disorder. In some embodiments, a method comprises the administration to a patient in need thereof of a therapeutically effective amount of a mesembrine composition for the treatment of anxiety and hysteria or anxiety and depression.

In some embodiments, pharmaceutical compositions reported herein can be provided in an oral dosage form. In some embodiments, an oral dosage form of mesembrine can be a capsule. In some embodiments, an oral dosage form of mesembrine is a tablet. In some embodiments, an oral dosage form comprises a filler. In some embodiments, an oral dosage form comprises two fillers. In some embodiments, an oral dosage form comprises one or more fillers. In some embodiments, an oral dosage form comprises one or more disintegrants. In some embodiments, the oral dosage form comprises one or more lubricants. In some embodiments, an oral dosage form comprises one or more glidants, anti-adherents and/or anti-statics. In some embodiments, an oral dosage form is prepared via dry blending. In some embodiments, an oral dosage form is a tablet and is prepared via dry granulation.

In some embodiments, a pharmaceutical composition comprises a solid form described herein; and a pharmaceutically acceptable excipient. In some embodiments, a pharmaceutical composition is formed by a process comprising dissolving a solid form described herein.

In some embodiments, a method of treating a mental health disorder, comprises administering to a mammal in need thereof an effective amount of a solid form described herein or a pharmaceutical composition described herein. In some embodiments, the mental health disorder is anxiety, stress, or depression. In some embodiments, the mammal is a human.

In some embodiments, a method of treating a mental health disorder, comprises administering to a mammal in need thereof an effective amount of a solid form of (−) mesembrine selected from:
a.) Freeform Form A having an X-ray powder diffraction (XRPD) pattern comprising 2-theta peaks (2 theta±0.2) at 19.8°;
b.) HCl Form A having an X-ray powder diffraction (XRPD) pattern comprising 2-theta peaks (2 theta±0.2) at 16.5°;
c.) Tartrate Form A having an X-ray powder diffraction (XRPD) pattern comprising 2-theta peaks (2 theta±0.2) at 20.7°;
d.) Besylate Form A having an X-ray powder diffraction (XRPD) pattern comprising 2-theta peaks (2 theta±0.2) at 9.3°;
e.) Besylate Form B having an X-ray powder diffraction (XRPD) pattern comprising 2-theta peaks (2 theta±0.2) at 23.9°;
f.) Oxalate Form A having an X-ray powder diffraction (XRPD) pattern comprising 2-theta peaks (2 theta±0.2) at 23.7°; and
g.) Gentisate Form A having an X-ray powder diffraction (XRPD) pattern comprising 2-theta peaks (2 theta±0.2) at 16.6°. In some embodiments, the recited XRPD peaks for each solid form are the most intense peaks by relative intensity in the respective XRPD patterns.

### Exemplary Embodiments

1. A solid Freeform Form A of mesembrine.
2. The solid form of embodiment 1, having an X-ray powder diffraction (XRPD) pattern comprising 2-theta peaks (2 theta±0.2) 12.9°, 13.9°, 14.9°, 19.8°, and 23.0°.
3. The solid form of embodiment 1, having an X-ray powder diffraction (XRPD) pattern comprising 2-theta peaks (2 theta±0.2) 7.4°, 12.9°, 13.9°, 14.9°, 17.5°, 19.0°, 19.8°, 23.0°, 25.4°, and 27.0°.
4. The solid form of embodiment 1, having an X-ray powder diffraction (XRPD) pattern comprising 2-theta peaks according to Table 4 having a relative intensity of greater than 10%.
5. The solid form of embodiment 1, having an X-ray powder diffraction (XRPD) pattern according to FIG. 1A.
6. The solid form of embodiment 1, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 12.9 | 6.9 |
| 13.9 | 6.4 |
| 14.9 | 5.9 |
| 19.8 | 4.5 |
| 23.0 | 3.9. |

7. The solid form of embodiment 1, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.4 | 11.9 |
| 12.9 | 6.9 |
| 13.9 | 6.4 |
| 14.9 | 5.9 |
| 17.5 | 5.1 |
| 19.0 | 4.7 |
| 19.8 | 4.5 |
| 23.0 | 3.9 |
| 25.4 | 3.5 |
| 27.0 | 3.3. |

8. The solid form of any one of embodiments 1-7, characterized by a TGA thermogram according to FIG. 1B.
9. The solid form of any one of embodiments 1-7, further characterized by a sample weight loss of up to about 3% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

10. The solid form of any one of embodiments 1-9, characterized by a differential scanning calorimetry (DSC) thermogram according to FIG. 1C.
11. The solid form of any one of embodiments 1-9, further characterized by a DSC having a broad endotherm at about 69.6° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

12. The solid form of any one of embodiments 1-11, characterized by a DVS isotherm plot according to FIG. 1E.
13. The solid form of any one of embodiments 1-12, characterized by an observed solubility of one or more of the following:
a) greater than about 8.3 mg/mL in H₂O;
b) greater than about 8.3 mg/mL in SGF;
c) greater than about 8.3 mg/mL in FaSSIF; or
d) greater than about 8.3 mg/mL in FeSSIF.
   14. A crystalline HCl Form A of mesembrine.
   15. The crystalline form of embodiment 14, having an X-ray powder diffraction (XRPD) pattern comprising 2-theta peaks (2 theta±0.2) 13.9°, 16.5°, 17.3°, 21.0°, and 27.3°.
   16. The crystalline form of embodiment 14, having an X-ray powder diffraction (XRPD) pattern comprising 2-theta peaks (2 theta±0.2) 10.8°, 12.1°, 12.6°, 13.9°, 15.9°, 16.5°, 17.3°, 17.7°, 18.9°, 20.2°, 21.0°, 21.3°, 21.7°, 22.1°, 23.1°, 24.2°, 24.5°, 25.1°, 25.4°, 26.3°, 26.7°, 27.3°, 27.8°, 28.2°, 29.5°, 31.5°, and 36.7°.
   17. The crystalline form of embodiment 14, having an X-ray powder diffraction (XRPD) pattern comprising 2-theta peaks according to Table 5 having a relative intensity of greater than 10%.
   18. The crystalline form of embodiment 14, having an X-ray powder diffraction (XRPD) pattern according to FIG. 2A.
   19. The crystalline form of embodiment 14, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 13.9 | 6.4 |
| 16.5 | 5.4 |
| 17.3 | 5.1 |
| 21.0 | 4.2 |
| 27.3 | 3.3. |

20. The crystalline form of embodiment 14, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 10.8 | 8.2 |
| 12.1 | 7.3 |
| 12.6 | 7.0 |
| 13.9 | 6.4 |
| 15.9 | 5.6 |
| 16.5 | 5.4 |
| 17.3 | 5.1 |
| 17.7 | 5.0 |
| 18.9 | 4.7 |
| 20.2 | 4.4 |
| 21.0 | 4.2 |
| 21.3 | 4.2 |
| 21.7 | 4.1 |
| 22.1 | 4.0 |
| 23.1 | 3.8 |
| 24.2 | 3.7 |
| 24.5 | 3.6 |
| 25.1 | 3.6 |
| 25.4 | 3.5 |
| 26.3 | 3.4 |
| 26.7 | 3.3 |
| 27.3 | 3.3 |
| 27.8 | 3.2 |
| 28.2 | 3.2 |
| 29.5 | 3.0 |
| 31.5 | 2.8 |
| 32.8 | 2.7 |
| 36.7 | 2.4. |

21. The crystalline form of any one of embodiments 14-20, characterized by a TGA thermogram according to FIG. 2B.
22. The crystalline form of any one of embodiments 14-20, further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

23. The crystalline form of any one of embodiments 14-22, characterized by a differential scanning calorimetry (DSC) thermogram according to FIG. 2C.
24. The crystalline form of any one of embodiments 14-22, further characterized by a DSC having an endotherm at about 210° C. to about 215° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

25. The crystalline form of any one of embodiments 14-24, characterized by substantially the same X-ray powder diffraction (XRPD) pattern of the bottom panel of FIG. 2D post storage at 40° C. and 75% RH for at least a week.
26. The crystalline form of any one of embodiments 14-25, characterized by a DVS isotherm plot according to FIG. 2E.
27. The crystalline form of any one of embodiments 14-26, characterized by substantially the same X-ray powder diffraction (XRPD) pre- and post-DVS according to FIG. 2F.
28. The crystalline form of any one of embodiments 14-27, having a water uptake at 25° C./80% RH of 0.72%.
29. The crystalline form of any one of embodiments 14-28, characterized by an observed solubility of one or more of the following:
a) greater than about 8.3 mg/mL in H₂O;
b) greater than about 8.3 mg/mL in SGF;
c) greater than about 8.3 mg/mL in FaSSIF; or
d) greater than about 8.3 mg/mL in FeSSIF.
   30. A crystalline Tartrate Form A of mesembrine.
   31. The crystalline form of embodiment 30, having an X-ray powder diffraction (XRPD) pattern comprising 2-theta peaks (2 theta±0.2) 5.2°, 10.3°, 12.9°, 16.7°, and 20.7°.
   32. The crystalline form of embodiment 30, having an X-ray powder diffraction (XRPD) pattern comprising 2-theta peaks (2 theta±0.2) 5.2°, 10.3°, 12.9°, 15.5°, 15.7°, 15.9°, 16.7°, 17.5°, 18.1°, 20.7°, 21.3°, 22.0°, 23.3°, 26.0°, and 26.2°.
   33. The crystalline form of embodiment 30, having an X-ray powder diffraction (XRPD) pattern comprising 2-theta peaks according to Table 6 having a relative intensity of greater than 10%.
   34 The crystalline form of embodiment 30, having an X-ray powder diffraction (XRPD) pattern according to FIG. 3A.
   35. The crystalline form of embodiment 30, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 5.2 | 17.1 |
| 10.3 | 8.6 |
| 12.9 | 6.9 |
| 16.7 | 5.3 |
| 20.7 | 4.3. |

36. The crystalline form of embodiment 30, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 5.2 | 17.1 |
| 10.3 | 8.6 |
| 12.9 | 6.9 |
| 15.5 | 5.7 |
| 15.7 | 5.6 |
| 15.9 | 5.6 |
| 16.7 | 5.3 |
| 17.5 | 5.1 |
| 18.1 | 4.9 |
| 20.7 | 4.3 |
| 21.3 | 4.2 |
| 22.0 | 4.0 |
| 23.3 | 3.8 |
| 26.0 | 3.4 |
| 26.2 | 3.4. |

37. The crystalline form of any one of embodiments 30-36, characterized by a TGA thermogram according to FIG. 3B.
38. The crystalline form of any one of embodiments 30-36, further characterized by a sample weight loss of up to about 3% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

39. The crystalline form of any one of embodiments 30-38, characterized by a differential scanning calorimetry (DSC) thermogram according to FIG. 3C.
40. The crystalline form of any one of embodiments 30-38, further characterized by a DSC having an endotherm at about 155° C. to about 160° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

41. A crystalline Besylate Form A of mesembrine.
42. The crystalline form of embodiment 41, having an X-ray powder diffraction (XRPD) pattern comprising 2-theta peaks (2 theta±0.2) 9.3°, 19.6°, 21.2°, 23.8°, and 24.7°.
43. The crystalline form of embodiment 41, having an X-ray powder diffraction (XRPD) pattern comprising 2-theta peaks (2 theta±0.2) 9.3°, 11.1°, 12.8°, 13.5°, 16.7°, 18.6°, 19.6°, 20.3°, 20.7°, 21.2°, 23.8°, 24.7°, and 28.0°.
44. The crystalline form of embodiment 41, having an X-ray powder diffraction (XRPD) pattern comprising 2-theta peaks according to Table 7 having a relative intensity of greater than 10%.
45. The crystalline form of embodiment 41, having an X-ray powder diffraction (XRPD) pattern according to FIG. 4A.
46. The crystalline form of embodiment 41, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.3 | 9.5 |
| 19.6 | 4.5 |
| 21.2 | 4.2 |
| 23.8 | 3.7 |
| 24.7 | 3.6. |

47. The crystalline form of embodiment 41, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.3 | 9.5 |
| 11.1 | 7.9 |
| 12.8 | 6.9 |
| 13.5 | 6.6 |
| 16.7 | 5.3 |
| 18.6 | 4.8 |
| 19.6 | 4.5 |
| 20.3 | 4.4 |
| 20.7 | 4.3 |
| 21.2 | 4.2 |
| 23.8 | 3.7 |
| 24.7 | 3.6 |
| 28.0 | 3.2. |

48. The crystalline form of any one of embodiments 41-47, characterized by a TGA thermogram according to FIG. 4B.
49. The crystalline form of any one of embodiments 41-47, further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

50. The crystalline form of any one of embodiments 41-49, characterized by a differential scanning calorimetry (DSC) thermogram according to FIG. 4C.
51. The crystalline form of any one of embodiments 41-49, further characterized by a DSC having an endotherm at about 163° C. to about 168° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

52. A crystalline Besylate Form B of mesembrine.
53. The crystalline form of embodiment 52, having an X-ray powder diffraction (XRPD) pattern comprising 2-theta peaks (2 theta±0.2) 11.0°, 13.5°, 15.2°, 18.7°, and 23.8°.
54. The crystalline form of embodiment 52, having an X-ray powder diffraction (XRPD) pattern comprising 2-theta peaks (2 theta±0.2) 7.5°, 9.4°, 11.0°, 11.7°, 12.6°, 13.5°, 15.2°, 15.7°, 16.2°, 16.8°, 18.0°, 18.3°, 18.7°, 18.9°, 21.5°, 21.9°, 22.5°, 22.8°, 23.4°, 23.8°, 24.2°, 24.8°, 25.2°, 26.4°, 26.7°, 28.1°, and 29.1°.
55. The crystalline form of embodiment 52, having an X-ray powder diffraction (XRPD) pattern comprising 2-theta peaks according to Table 8 having a relative intensity of greater than 10%.
56. The crystalline form of embodiment 52, having an X-ray powder diffraction (XRPD) pattern according to FIG. 5A.
57. The crystalline form of embodiment 52, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 11.0 | 8.0 |
| 13.5 | 6.6 |
| 15.2 | 5.8 |
| 18.7 | 4.7 |
| 23.8 | 3.7. |

58. The crystalline form of embodiment 52, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.5 | 11.8 |
| 9.4 | 9.4 |
| 11.0 | 8.0 |
| 11.7 | 7.6 |
| 12.6 | 7.0 |
| 13.5 | 6.6 |
| 15.2 | 5.8 |
| 15.7 | 5.6 |
| 16.2 | 5.5 |
| 16.8 | 5.3 |
| 18.0 | 4.9 |
| 18.3 | 4.9 |
| 18.7 | 4.7 |
| 18.9 | 4.7 |
| 21.5 | 4.1 |
| 21.9 | 4.1 |
| 22.5 | 4.0 |
| 22.8 | 3.9 |
| 23.4 | 3.8 |
| 23.8 | 3.7 |
| 24.2 | 3.7 |
| 24.8 | 3.6 |
| 25.2 | 3.5 |
| 26.4 | 3.4 |
| 26.7 | 3.3 |
| 28.1 | 3.2 |
| 29.1 | 3.1. |

59. The crystalline form of any one of embodiments 52-58, characterized by a TGA thermogram according to FIG. 5B.
60. The crystalline form of any one of embodiments 52-58, further characterized by a sample weight loss of up to about 1% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

61. The crystalline form of any one of embodiments 52-60, characterized by a differential scanning calorimetry (DSC) thermogram according to FIG. 5C.
62. The crystalline form of any one of embodiments 52-60, further characterized by a DSC having endotherm at about 183° C. to about 188° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

63. The crystalline form of any one of embodiments 52-62, characterized by substantially the same X-ray powder diffraction (XRPD) pattern of the bottom panel of FIG. 5D post storage at 40° C. and 75% RH for at least a week.
64. The crystalline form of any one of embodiments 52-63, characterized by a DVS isotherm plot according to FIG. 5E.
65. The crystalline form of any one of embodiments 52-64, characterized by substantially the same X-ray powder diffraction (XRPD) pre- and post-DVS according to FIG. 5F.
66. The crystalline form of any one of embodiments 52-65, having a water uptake at 25° C./80% RH of 0.32%.
67. The crystalline form of any one of embodiments 52-66, characterized by an observed solubility of one or more of the following:
a) greater than about 8.3 mg/mL in H₂O;
b) greater than about 8.3 mg/mL in SGF;
c) greater than about 8.3 mg/mL in FaSSIF; or
d) greater than about 8.3 mg/mL in FeSSIF.
   68. A crystalline Oxalate Form A of mesembrine.
   69. The crystalline form of embodiment 68, having an X-ray powder diffraction (XRPD) pattern comprising 2-theta peaks (2 theta±0.2) 7.9°, 11.7°, 17.3°, 18.2°, and 23.7°.
   70. The crystalline form of embodiment 68, having an X-ray powder diffraction (XRPD) pattern comprising 2-theta peaks (2 theta±0.2) 7.9°, 11.3°, 11.7°, 12.2°, 13.8°, 13.9°, 14.6°, 15.8°, 16.5, 17.2°, 17.3°, 17.6°, 18.0°, 18.2°, 19.4°, 20.6°, 22.8°, 23.0°, 23.4°, 23.7°, 24.0°, 24.3°, 24.6°, 25.3°, 25.5°, 26.0°, 26.4°, 26.6°, 30.1°, and 32.6°.
   71. The crystalline form of embodiment 68, having an X-ray powder diffraction (XRPD) pattern comprising 2-theta peaks according to Table 9 having a relative intensity of greater than 10%.
   72. The crystalline form of embodiment 68, having an X-ray powder diffraction (XRPD) pattern according to FIG. 6A.
   73. The crystalline form of embodiment 68, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.9 | 11.2 |
| 11.7 | 7.6 |
| 17.3 | 5.1 |
| 18.2 | 4.9 |
| 23.7 | 3.8. |

74. The crystalline form of embodiment 68, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 7.9 | 11.2 |
| 11.3 | 7.8 |
| 11.7 | 7.6 |
| 12.2 | 7.3 |
| 13.8 | 6.4 |
| 13.9 | 6.4 |
| 14.6 | 6.1 |
| 15.8 | 5.6 |
| 16.5 | 5.4 |
| 17.2 | 5.2 |
| 17.3 | 5.1 |
| 17.6 | 5.0 |
| 18.0 | 4.9 |
| 18.2 | 4.9 |
| 19.4 | 4.6 |
| 20.6 | 4.3 |
| 22.8 | 3.9 |
| 23.0 | 3.9 |
| 23.4 | 3.8 |
| 23.7 | 3.8 |
| 24.0 | 3.7 |
| 24.3 | 3.7 |
| 24.6 | 3.6 |
| 25.3 | 3.5 |
| 25.5 | 3.5 |
| 26.0 | 3.4 |
| 26.4 | 3.4 |
| 26.6 | 3.4 |
| 30.1 | 3.0 |
| 32.6 | 2.8. |

75. The crystalline form of any one of embodiments 68-74, characterized by a TGA thermogram according to FIG. 6B.
76. The crystalline form of any one of embodiments 68-74, further characterized by a sample weight loss of up to about 3.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

77. The crystalline form of any one of embodiments 68-76, characterized by a differential scanning calorimetry (DSC) thermogram according to FIG. 6C.
78. The crystalline form of any one of embodiments 68-76, further characterized by a DSC having endotherm at about 150° C. to about 155° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

79. A crystalline Gentisate Form A of mesembrine.
80. The crystalline form of embodiment 79, having an X-ray powder diffraction (XRPD) pattern comprising 2-theta peaks (2 theta±0.2) 13.8°, 16.6°, 19.2°, 20.8°, and 27.8°.
81. The crystalline form of embodiment 79, having an X-ray powder diffraction (XRPD) pattern comprising 2-theta peaks (2 theta±0.2) 8.9°, 11.4°, 12.8°, 13.2°, 13.8°, 16.1°, 16.6°, 16.9°, 17.2°, 19.2°, 19.5°, 20.4°, 20.8°, 21.0°, 21.6°, 22.1°, 24.2°, 24.4°, 24.9°, 25.2°, 26.7°, 27.8°, and 29.1°.
82. The crystalline form of embodiment 79, having an X-ray powder diffraction (XRPD) pattern comprising 2-theta peaks according to Table 10 having a relative intensity of greater than 10%.
83. The crystalline form of embodiment 79, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 13.8 | 6.4 |
| 16.6 | 5.4 |
| 19.2 | 4.6 |
| 20.8 | 4.3 |
| 27.8 | 3.2. |

84. The crystalline form of embodiment 79, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 8.9 | 9.9 |
| 11.4 | 7.8 |
| 12.8 | 6.9 |
| 13.2 | 6.7 |
| 13.8 | 6.4 |
| 16.1 | 5.5 |
| 16.6 | 5.4 |
| 16.9 | 5.2 |
| 17.2 | 5.2 |
| 19.2 | 4.6 |
| 19.5 | 4.6 |
| 20.4 | 4.4 |
| 20.8 | 4.3 |
| 21.0 | 4.2 |
| 21.6 | 4.1 |
| 22.1 | 4.0 |
| 24.2 | 3.7 |
| 24.4 | 3.6 |
| 24.9 | 3.6 |
| 25.2 | 3.5 |
| 26.7 | 3.3 |
| 27.8 | 3.2 |
| 29.1 | 3.1. |

85. The crystalline form of embodiment 79, having an X-ray powder diffraction (XRPD) pattern according to FIG. 7A.
86. The crystalline form of any one of embodiments 79-85, characterized by a TGA thermogram according to FIG. 7B.
87. The crystalline form of any one of embodiments 79-85, further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂. |

88. The crystalline form of any one of embodiments 79-87, characterized by a differential scanning calorimetry (DSC) thermogram according to FIG. 7C.
89. The crystalline form of any one of embodiments 79-87, further characterized by a DSC having an endotherm at about 198° C. to about 203° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

90. The crystalline form of any one of embodiments 79-89, characterized by substantially the same X-ray powder diffraction (XRPD) pattern of the bottom panel of FIG. 7D post storage at 40° C. and 75% RH for at least a week.
91. The crystalline form of any one of embodiments 79-90, characterized by a DVS isotherm plot according to FIG. 7E.
92. The crystalline form of any one of embodiments 79-91, characterized by substantially the same X-ray powder diffraction (XRPD) pre- and post-DVS according to FIG. 7F.
93. The crystalline form of any one of embodiments 79-92, having a water uptake at 25° C./80% RH of 0.17%.
94. The crystalline form of any one of embodiments 79-93, characterized by an observed solubility of one or more of the following:
a) greater than about 3 mg/mL in H₂O;
b) greater than about 5.5 mg/mL in SGF;
c) greater than about 3.5 mg/mL in FaSSIF; or
d) greater than about 4 mg/mL in FeSSIF.
   95. A pharmaceutical composition, comprising a solid form of any one of embodiments 1-94; and a pharmaceutically acceptable excipient.
   96. A process for preparing a pharmaceutical composition, comprising dissolving in a solvent a solid form any one of embodiments 1-94.
   97. A method of treating a mental health disorder, comprising administering to a mammal in need thereof an effective amount of a solid form according to any one of embodiments 1-94 or a composition of claim 95 or 96.
   98. The method of embodiment 97, wherein the mental health disorder is anxiety, stress, or depression.
   99. The method of embodiment 97, wherein the mental health disorder is anxiety.
   100. The method of embodiment 97, wherein the mental health disorder is stress.
   101. The method of embodiment 97, wherein the mental health disorder is depression.
   102. The method of any one of embodiments 97-101, wherein the mammal is a human.
   103. A composition comprising a solid form of mesembrine in combination with one or more compounds selected from the group consisting of hydrochloric acid, tartaric acid, oxalic acid, benzensulfonic acid, and gentisic acid.
   104. The composition of embodiment 103, wherein the mesembrine is (−) mesembrine.
   105. A solid form produced by a process of:
1.) dissolving a free base of (−) mesembrine in acetone to form a (−) mesembrine solution;
2.) adding hydrochloric acid to the (−) mesembrine solution; and
3.) isolating the solids formed in step (2),
   wherein the isolated solids:
a) show a weight loss of about 1% to about 1.5% up to 150° C. by TGA; and/or
b) show an endotherm at about 210° C. to about 215° C. (peak temperature) by DSC.
   106. A solid form produced by a process of:
1.) dissolving a free base of (−) mesembrine in ethanol to form a (−) mesembrine solution;
2.) adding 1-tartaric acid to the (−) mesembrine solution; and
3.) isolating the solids formed in step (2),
   wherein the isolated solids:
a) show a weight loss of about 2% to about 3% up to 150° C. by TGA; and/or
b) show an endotherm at about 155° C. to about 160° C. (peak temperature) by DSC.
   107. A solid form produced by a process of:
1.) dissolving a free base of (−) mesembrine in acetone to form a (−) mesembrine solution;
2.) adding benzenesulfonic acid to the (−) mesembrine solution; and
3.) isolating the solids formed in step (2),
   wherein the isolated solids:
a) show a weight loss of about 1% to about 1.5% up to 150° C. by TGA; and/or
b) show an endotherm at about 163° C. to about 168° C. (peak temperature) by DSC.
   108. A solid form produced by a process of:
1.) combining a free base of (−) mesembrine and benzenesulfonic acid;
2.) adding acetone to the mixture of step (1); and
3.) isolating the solids formed in step (2),
   wherein the isolated solids:
a) show a weight loss of about 0.5% to about 1% up to 150° C. by TGA; and/or
b) show an endotherm at about 183° C. to about 188° C. (peak temperature) by DSC.
   109. A solid form produced by a process of:
1.) dissolving a free base of (−) mesembrine in acetone to form a (−) mesembrine solution;
2.) adding oxalic acid to the (−) mesembrine solution; and
3.) isolating the solids formed in step (2),
   wherein the isolated solids:
a) show a weight loss of about 3% to about 3.5% up to 150° C. by TGA; and/or
b) show an endotherm at about 150° C. to about 155° C. (peak temperature) by DSC.
   110. A solid form produced by a process of:
1.) combining a free base of (−) mesembrine and gentisic acid;
2.) adding ethanol to the mixture of step (1); and
3.) isolating the solids formed in step (2),
   wherein the isolated solids:
a) show a weight loss of about 1% to about 1.5% up to 150° C. by TGA; and/or
b) show an endotherm at about 198° C. to about 203° C. (peak temperature) by DSC.
   111. A method of treating a mental health disorder, comprising administering to a mammal in need thereof an effective amount of a solid form of (−) mesembrine selected from:
a.) Freeform Form A having an X-ray powder diffraction (XRPD) pattern comprising 2-theta peaks (2 theta±0.2) at 19.8°;
b.) HCl Form A having an X-ray powder diffraction (XRPD) pattern comprising 2-theta peaks (2 theta±0.2) at 16.5°;
c.) Tartrate Form A having an X-ray powder diffraction (XRPD) pattern comprising 2-theta peaks (2 theta±0.2) at 20.7°;
d.) Besylate Form A having an X-ray powder diffraction (XRPD) pattern comprising 2-theta peaks (2 theta±0.2) at 9.3°;
e.) Besylate Form B having an X-ray powder diffraction (XRPD) pattern comprising 2-theta peaks (2 theta±0.2) at 23.9°;
f.) Oxalate Form A having an X-ray powder diffraction (XRPD) pattern comprising 2-theta peaks (2 theta±0.2) at 23.7°; and
g.) Gentisate Form A having an X-ray powder diffraction (XRPD) pattern comprising 2-theta peaks (2 theta±0.2) at 16.6°.
   112. The method of embodiment 83, wherein the recited XRPD peaks for each solid form are the most intense peaks by relative intensity in the respective XRPD patterns.

### EXAMPLES

### Instruments and Methods

### XRPD

For XRPD analysis, Empyrean X-ray powder diffractometer from Malvern Panalytical was used. Sample was spread on the middle of a zero-background Si holder. The XRPD parameters used are listed in Table 1.

TABLE 1
Parameters for XRPD test

| | |
|---|---|
| Parameters | Reflection Mode |
| Model | Empyrean |
| X-Ray wavelength | Cu, kα, |
| | Kα1 (Å): 1.540598, Kα2 |
| | (Å): 1.544426 |
| | Kα2/Kα1 intensity ratio: |
| | 0.50 |
| X-Ray tube setting | 45 kV, 40 mA |
| Divergence slit | ⅛° |
| Scan mode | Continuous |
| Scan range (°2TH) | 3°-40° |
| Scan step time (s) | 46.67 |
| Step size (°2TH) | 0.0263 |
| Test time | 5 min 4 s |

### TGA

TGA data were collected using a TA Discovery TGA 5500 TGA from TA Instruments. Detailed parameters used are listed in Table 2.

TABLE 2
Parameters for TGA test

| | |
|---|---|
| Parameters | TGA |
| Method | Ramp |
| Sample pan | Aluminum, open |
| Temperature | RT-350° C. |
| Heating rate | 10° C./min |
| Purge gas | N₂ |

### DSC

DSC data were collected using a TA Discovery DSC 2500 DSC from TA Instruments. Detailed parameters used are listed in Table 3 Error! Reference source not found.

TABLE 3
Parameters for DSC test

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

### Purity HPLC Conditions

| Instrument | Agilent 1260 with DAD detector | |
|---|---|---|
| Column | Waters Xbridge C18, 4.6*150 mm, | |
| | 5 μm | |
| Mobile phase | A: 0.0375% TFA in H₂O | |
| | B: 0.01875% TFA in ACN | |
| | Time (min) | % B |
| Gradient | 0.0 | 5 |
| | 2.0 | 5 |
| | 12.0 | 30 |
| | 20.0 | 90 |
| | 22.0 | 90 |
| | 22.1 | 5 |
| | 28.0 | 5 |

| | |
|---|---|
| Flow rate | 1.0 mL/min |
| Injection volume | 5 μL |
| Detector wavelength | 230 nm |
| Column temperature | 40° C. |
| Sampler temperature | RT |
| Diluent | ACN/H₂O (1:1, v/v) |

### Solubility HPLC Conditions

| Instrument | Agilent 1260 with DAD detector | |
|---|---|---|
| Column | Waters Xbridge C18, 4.6*150 mm, 5 | |
| | μm | |
| Mobile phase | A: 0.0375% TFA in H₂O | |
| | B: 0.01875% TFA in ACN | |
| | Time (min) | % B |
| Gradient | 0.0 | 5 |
| | 4.0 | 30 |
| | 6.0 | 90 |
| | 8.0 | 90 |
| | 8.1 | 5 |
| | 12.0 | 5 |

| | |
|---|---|
| Flow rate | 1.0 mL/min |
| Injection volume | 5 μL |
| Detector wavelength | 230 nm |
| Column temperature | 40° C. |
| Sampler temperature | RT |
| Diluent | ACN/H₂O (1:1, v/v) |

### Mesembrine Synthesis

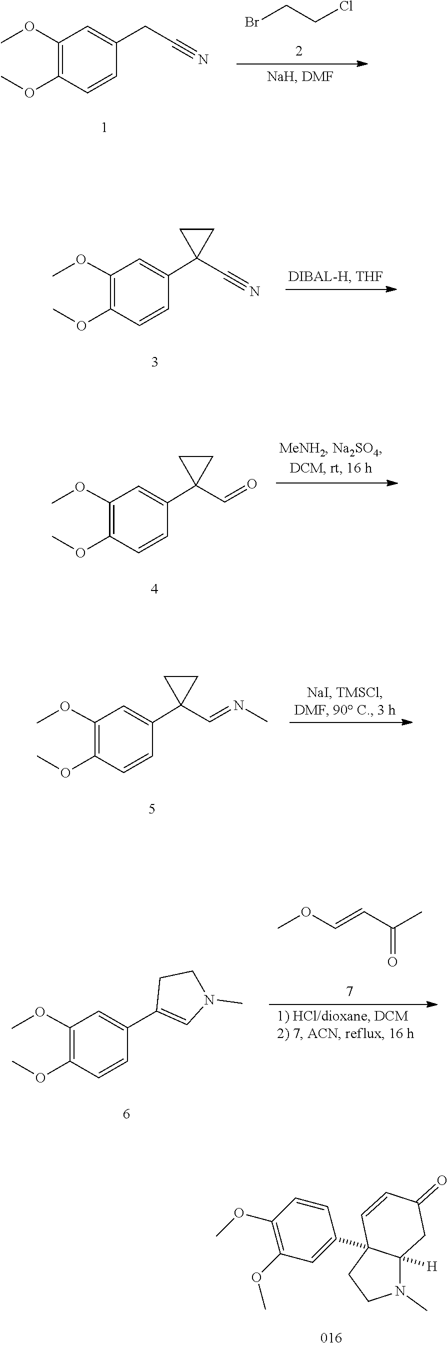

### Step 1—1-(3,4-dimethoxyphenyl)cyclopropanecarbonitrile (3)

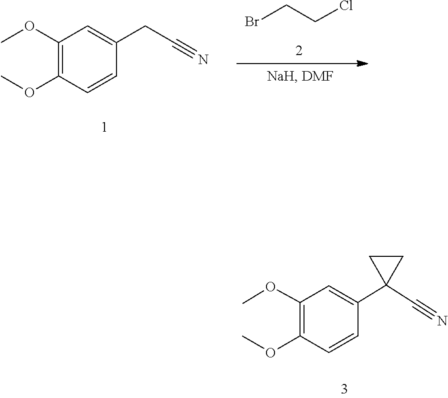

A mixture of 2-(3,4-dimethoxyphenyl) acetonitrile (20 g, 112 mmol, CAS #93-17-4) in DMF (93 mL) was added NaH (18.0 g, 451 mmol, 60% purity) in portions and the mixture was stirred at 25° C. for 20 minutes. 1-bromo-2-chloro-ethane (16.1 g, 112 mmol, CAS #107-04-0) was added, and then mixture stirred at 25° C. for 16 hours. On completion, the reaction was quenched by methanol/water mixture (1:1, 1000 mL) and the reaction products were extracted into ethyl acetate (3×500 mL). The combined extracts were washed with water (4×500 mL), brine (1×200 mL) and then dried (Na₂SO₄). The solvent was then removed under reduced pressure to give residue. The residue was purified by column chromatography (SiO₂, Petroleum ether/Ethyl acetate=10/1 to 3/1) to give the title compound (15 g, 65% yield) as yellow oil.

¹H NMR (400 MHZ, CHLOROFORM-d) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2—1-(3,4-dimethoxyphenyl)cyclopropanecarbaldehyde (4)

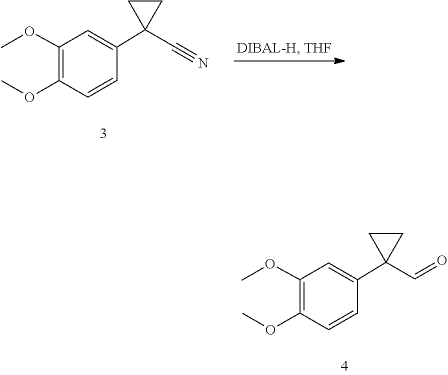

To a solution of 1-(3,4-dimethoxyphenyl)cyclopropanecarbonitrile (11 g, 54.1 mmol) in THF (160 mL) was added DIBAL-H (1 M, 81.19 mL). The mixture was stirred at 25° C. for 3 hours. On completion, the reaction was cautiously quenched by addition of 2 M HCl aqueous soliton, extracted with dichloromethane (3×200 mL). The combined extracts were washed with water (2×200 mL), brine (2×200 mL) and then dried (Na₂SO₄) to give the title compound (9.6 g, 85% yield) as yellow oil.

LC-MS (ESI+) m/z 207.0 (M+H)⁺

¹H NMR (400 MHZ, CHLOROFORM-d) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3—(Z)-1-[1-(3,4-dimethoxyphenyl)cyclopropyl]-N-methyl-methanimine (5)

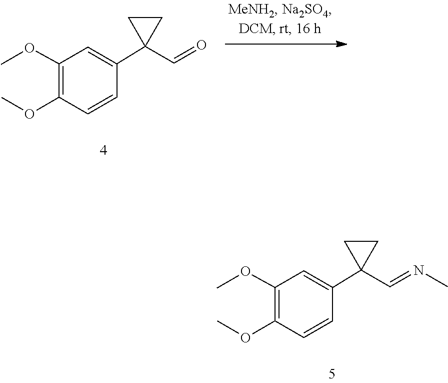

To a solution of 1-(3,4-dimethoxyphenyl)cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was stirred at 25° C. for 16 hours. On completion, the reaction mixture was filtered and the filtrate was concentrated under reduced pressure to give the title compound (5.1 g, 99% yield) as white solid. LC-MS (ESI⁺) m/z 219.9 (M+H)⁺, major Int.4 mass on LCMS;

¹H NMR (400 MHZ, CHLOROFORM-d) δ 7.55 (q, J=1.2 Hz, 1H), 6.93-6.77 (m, 3H), 3.88 (d, J=7.2 Hz, 6H), 3.24 (d, J=1.6 Hz, 3H), 1.29-1.23 (m, 2H), 1.18-1.12 (m, 2H).

### Step 4—4-(3,4-dimethoxyphenyl)-1-methyl-2,3-dihydropyrrole (6)

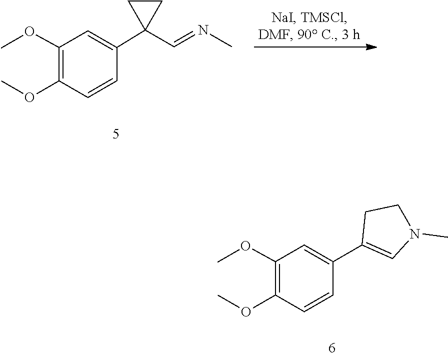

To a solution of (Z)-1-[1-(3,4-dimethoxyphenyl)cyclopropyl]-N-methyl-methanimine (5.4 g, 24.6 mmol) in DMF (19 mL) was added NaI (366 mg, 2.44 mmol) and TMSCl (267 mg, 2.46 mmol). The mixture was stirred at 90° C. for 3 hours. On completion, the reaction mixture was diluted with water (100 mL) and extracted with EtOAc (100 mL×3). The organic phase was wash with water and brine, dried over with anhydrous sodium sulfate, concentrated under reduced pressure to give the title compound (6.25 g, 80% yield) as yellow oil.

LC-MS (ESI⁺) m/z 220.0 (M+H)⁺.

¹H NMR (400 MHZ, CHLOROFORM-d) δ 6.90-6.66 (m, 3H), 6.31 (t, J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5—3a-(3,4-dimethoxyphenyl)-1-methyl-2,3,7,7a-tetrahydroindol-6-one (016)

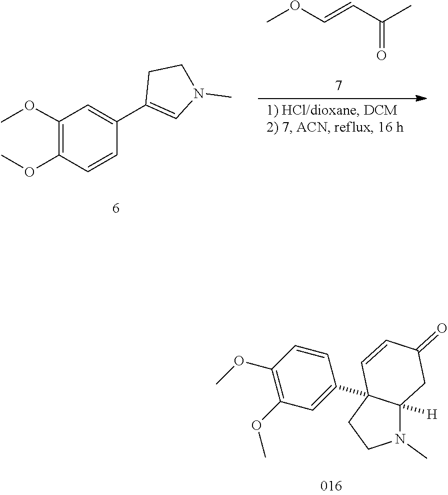

4-(3,4-dimethoxyphenyl)-1-methyl-2,3-dihydropyrrole (6.25 g, 28.5 mmol) was dissolved in dichloromethane (100 mL). To this was added HCl/dioxane (25 mL of a 1.0 M solution, 100 mmol) and the crude HCl salt was evaporated to dryness. A solution of obtained HCl slat, (E)-4-methoxybut-3-en-2-one (4.28 g, 42.7 mmol, CAS #4652-27-1) in ACN (90 mL) was stirred at 90° C. for 16 hours. On completion, the reaction mixture was filtered and the filtrate was concentrated under reduced pressure to give a residue, the residue was purified by prep-HPLC (column: Phenomenex luna C18 (250*70 mm, 10 um); mobile phase: [water (NH₄HCO₃)-ACN]; B %: 22%-52%,20 min) and acidified with aq. HCl to give desired compound (3.0 g, 30% yield) as a white solid.

LC-MS (ESI+) m z 288.3 (M+H)⁺.

¹H NMR (400 MHZ, CHLOROFORM-d) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6 (3aR,7aR)-3a-(3,4-dimethoxyphenyl)-1-methyloctahydro-6H-indol-6-one (022)

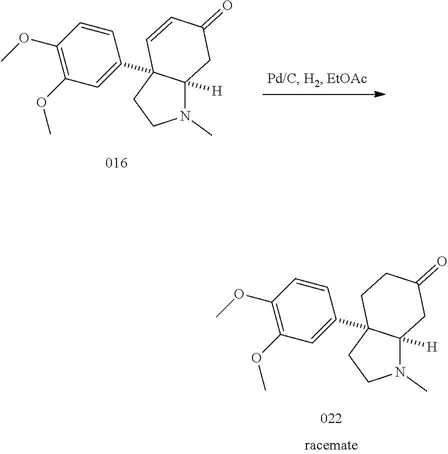

To a mixture of 3a-(3,4-dimethoxyphenyl)-2,3,7,7a-tetrahydro-1H-indol-6-one (12.0 g, 43.9 mmol, 016), Pd/C (300 mg, 4.39 mmol, 10% purity) in EtOAc (120 mL) was degassed and purged with H₂ for 3 times, and then the mixture was stirred at 25° C. for 2 hours under H₂ atmosphere (15 psi). On completion, the reaction mixture was filtered and concentrated in vacuo to give the title compound (10 g, 80% yield) as brown oil.

LC-MS (ESI+) m/z 290.4 (M+H)+

¹H NMR (400 MHZ, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Step 6 Chiral Resolution (001)

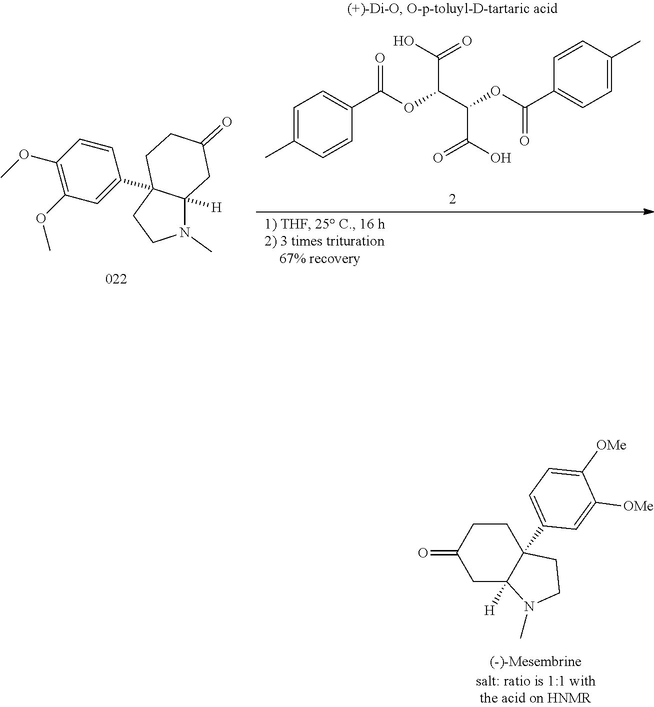

To a solution of 3a-(3,4-dimethoxyphenyl)-1-methyl-2,3,4,5,7,7a-hexahydroindol-6-one (28.0 g, 85.1 mmol) in THF (1400 mL, 50 V) was added (2S,3S)-2,3-bis [(4-methylbenzoyl)oxy]butanedioic acid (19.7 g, 51.1 mmol, CAS: 32634-68-7). The obtained suspension was stirred at 25° C. for 16 hours and then filtered. The solid was dried under vacuo and collected to give (3aS,7aS)-3a-(3,4-dimethoxyphenyl)-1-methyl-2,3,4,5,7,7a-hexahydroindol-6-one (25.0 g, 98% purity, 72% de, salt with acid 2) as a white solid. Then the obtained solid was triturated with THF (30 V each for 3 times) at 25° C. for 16 hours to give (3aS,7aS)-3a-(3,4-dimethoxyphenyl)-1-methyl-2,3,4,5,7,7a-hexahydroindol-6-one (22 g, 98% purity, 95% de, salt with acid 2) as a white solid.

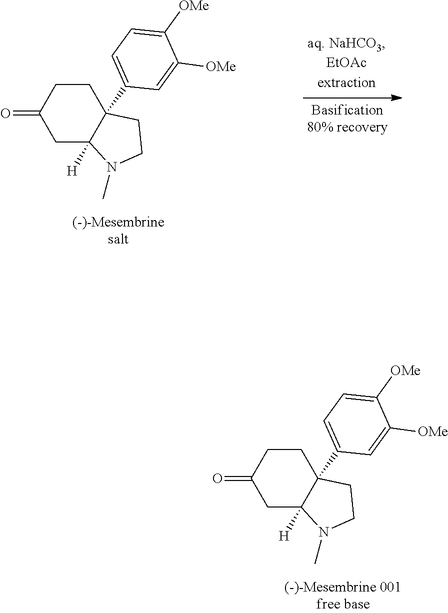 [SMILES: COc1ccc([C@@]23CCC(=O)C[C@@H]2N(C)CC3)cc1OC ; COc1ccc([C@@]23CCC(=O)C[C@@H]2N(C)CC3)cc1OC]

(3aS,7aS)-3a-(3,4-dimethoxyphenyl)-1-methyl-2,3,4,5,7,7a-hexahydroindol-6-one (22 g, salt) was poured to the saturated sodium bicarbonate solution (500 mL) and extracted with ethyl acetate (500 mL). The organic layers was dried by sodium sulfate, filtered and concentrated in vacuo to give the (3aS,7aS)-3a-(3,4-dimethoxyphenyl)-1-methyl-2,3,4,5,7,7a-hexahydroindol-6-one (7.50 g, 95% purity, 95% ee, free base) as a yellow gum.

LC-MS (ESI⁺) m/z 290.6 (M+H)⁺

¹H NMR (400 MHZ, CDCl₃) δ 6.89-6.81 (m, 2H), 6.80-6.75 (m, 1H), 3.82 (d, J=8.0 Hz, 6H), 3.11-3.03 (m, 1H), 2.88 (t, J=3.6 Hz, 1H), 2.59-2.48 (m, 2H), 2.43-2.32 (m, 1H), 2.31-2.21 (m, 4H), 2.20-2.09 (m, 3H), 2.08 (br s, 2H).

### Example 1—Freeform Form A

Freeform Form A was a gum material. The material had the solubility shown below:

| Solvent | Solubility (mg/mL) | Solvent | Solubility (mg/mL) |
|---|---|---|---|
| MeOH | S > 36.0 | THF | S > 44.0 |
| EtOH | S > 42.0 | 2-MeTHF | S > 40.0 |
| IPA | S > 38.0 | CPME | S > 40.0 |
| Acetone | S > 40.0 | 1,4-Dioxane | S > 40.0 |
| MIBK | S > 38.0 | ACN | S > 44.0 |
| EtOAc | 19.0 < S < 38.0 | DCM | S > 42.0 |
| IPAc | S > 36.0 | DMSO | S > 42.0.0 |
| MTBE | S > 40.0 | H₂O | S > 42.0 |

The solid was analyzed by XRPD, TGA, and DSC as described above. The results are shown in FIGS. 1A-1C. By TGA, the sample showed weight loss of 3.14% up to 150° C. DSC showed a broad endotherm at 69.6° C. (peak temperature).

TABLE 4
Diffraction peaks of Freeform Form A:

| #Peak | 2θ[°] | d-spacing [Å] | Rel. Int. [%] |
|---|---|---|---|
| 1 | 7.45 | 11.87 | 14.31 |
| 2 | 12.90 | 6.86 | 50.95 |
| 3 | 13.93 | 6.36 | 39.61 |
| 4 | 14.91 | 5.94 | 40.25 |
| 5 | 17.47 | 5.08 | 13.79 |
| 6 | 19.02 | 4.67 | 22.66 |
| 7 | 19.75 | 4.49 | 100.00 |
| 8 | 22.42 | 3.97 | 6.69 |
| 9 | 23.04 | 3.86 | 37.12 |
| 10 | 24.78 | 3.59 | 4.34 |
| 11 | 25.38 | 3.51 | 23.36 |
| 12 | 25.94 | 3.43 | 7.02 |
| 13 | 26.99 | 3.30 | 11.84 |
| 14 | 28.55 | 3.13 | 7.80 |
| 15 | 29.54 | 3.02 | 7.01 |
| 16 | 30.03 | 2.98 | 7.06 |
| 17 | 32.78 | 2.73 | 6.31 |
| 18 | 34.93 | 2.57 | 6.26 |
| 19 | 36.60 | 2.46 | 2.88 |

### Example 2—HCl Form A

### Preparation Method:

1. Freeform Form A (300.3 mg) was weighed into a 20 mL glass vial. Acetone (6 mL) was added to dissolve Freeform Form A solids.
2. The solution was treated with hydrochloric acid (38%, 87 uL) at room temperature.
3. The mixture was magnetically stirred at RT for 2 days.
4. The suspension was centrifuged (10000 rpm, 2 min) and solids were dried solid at RT under vacuum for 3 days.

The resulting solid was analyzed by XRPD, TGA, and DSC as described above. The results are shown in FIGS. 2A-2C. By TGA, the sample showed weight loss of 1.37% up to 150° C. DSC showed an endotherm at 213.7° C. (peak temperature).

TABLE 5
Diffraction peaks of HCl salt Form A

| #Peak | 2θ[°] | d-spacing [Å] | Rel. Int. [%] |
|---|---|---|---|
| 1 | 10.82 | 8.18 | 14.87 |
| 2 | 12.06 | 7.34 | 24.38 |
| 3 | 12.58 | 7.04 | 17.28 |
| 4 | 13.94 | 6.35 | 54.56 |
| 5 | 15.92 | 5.57 | 25.35 |
| 6 | 16.50 | 5.37 | 100.00 |
| 7 | 17.34 | 5.11 | 86.96 |
| 8 | 17.71 | 5.01 | 79.09 |
| 9 | 17.99 | 4.93 | 5.24 |
| 10 | 18.91 | 4.69 | 15.89 |
| 11 | 20.24 | 4.39 | 47.52 |
| 12 | 21.03 | 4.22 | 69.98 |
| 13 | 21.33 | 4.17 | 28.22 |
| 14 | 21.71 | 4.09 | 19.89 |
| 15 | 22.11 | 4.02 | 52.01 |
| 16 | 22.67 | 3.92 | 7.61 |
| 17 | 23.12 | 3.85 | 33.04 |
| 18 | 24.24 | 3.67 | 14.84 |
| 19 | 24.49 | 3.64 | 22.49 |
| 20 | 25.08 | 3.55 | 26.94 |
| 21 | 25.43 | 3.50 | 12.67 |
| 22 | 26.05 | 3.42 | 8.91 |
| 23 | 26.31 | 3.39 | 12.48 |
| 24 | 26.72 | 3.34 | 12.26 |
| 25 | 27.29 | 3.27 | 54.06 |
| 26 | 27.82 | 3.21 | 25.94 |
| 27 | 28.24 | 3.16 | 45.32 |
| 28 | 28.94 | 3.09 | 8.53 |
| 29 | 29.54 | 3.02 | 10.77 |
| 30 | 29.76 | 3.00 | 8.55 |
| 31 | 30.50 | 2.93 | 8.70 |
| 32 | 31.49 | 2.84 | 10.00 |
| 33 | 32.85 | 2.73 | 9.26 |
| 34 | 33.30 | 2.69 | 3.64 |
| 35 | 33.91 | 2.64 | 7.32 |
| 36 | 34.19 | 2.62 | 3.65 |
| 37 | 34.55 | 2.60 | 3.94 |
| 38 | 35.05 | 2.56 | 6.39 |
| 39 | 35.81 | 2.51 | 2.05 |
| 40 | 36.25 | 2.48 | 6.06 |
| 41 | 36.67 | 2.45 | 13.40 |
| 42 | 37.18 | 2.42 | 4.74 |
| 43 | 38.42 | 2.34 | 1.82 |
| 44 | 38.85 | 2.32 | 2.24 |
| 45 | 39.43 | 2.29 | 0.82 |

### Example 3—Tartrate Form A

### Preparation Method:

1. Freeform Form A (560.1 mg) was weighed into a 20 mL glass vial. Ethanol (14 mL) was added to dissolve Freeform Form A solids.
2. L-tartaric acid (10.4 mg) was weighed into an HPLC vial. Ethanol solution of Freeform Form A (0.5 mL) was added into the vial.
3. The mixture was magnetically stirred at room temperature (RT) for 3 days.
4. The suspension was centrifuged (10000 rpm, 2 min) and solids were dried solid at RT under vacuum for 1 day.

The resulting solid was analyzed by XRPD, TGA, and DSC as described above. The results are shown in FIGS. 3A-3C. By TGA, the sample showed weight loss of 2.79% up to 150° C. DSC showed an endotherm at 156.7° C. (peak temperature).

TABLE 6
Diffraction peaks of Tartrate Form A

| #Peak | 2θ[°] | d-spacing [Å] | Rel. Int. [%] |
|---|---|---|---|
| 1 | 5.18 | 17.06 | 47.30 |
| 2 | 7.72 | 11.45 | 3.07 |
| 3 | 10.34 | 8.56 | 93.88 |
| 4 | 11.48 | 7.71 | 4.82 |
| 5 | 12.10 | 7.32 | 4.24 |
| 6 | 12.91 | 6.86 | 31.27 |
| 7 | 14.14 | 6.26 | 5.92 |
| 8 | 14.69 | 6.03 | 3.19 |
| 9 | 15.49 | 5.72 | 28.61 |
| 10 | 15.72 | 5.64 | 27.52 |
| 11 | 15.94 | 5.56 | 16.66 |
| 12 | 16.66 | 5.32 | 37.06 |
| 13 | 17.54 | 5.06 | 11.24 |
| 14 | 18.10 | 4.90 | 20.62 |
| 15 | 18.74 | 4.73 | 8.87 |
| 16 | 19.52 | 4.55 | 5.57 |
| 17 | 20.73 | 4.29 | 100.00 |
| 18 | 21.30 | 4.17 | 13.72 |
| 19 | 21.99 | 4.04 | 9.98 |
| 20 | 23.34 | 3.81 | 10.12 |
| 21 | 23.98 | 3.71 | 4.14 |
| 22 | 24.55 | 3.63 | 3.72 |
| 23 | 25.36 | 3.51 | 8.00 |
| 24 | 25.95 | 3.43 | 17.38 |
| 25 | 26.22 | 3.40 | 13.66 |
| 26 | 27.79 | 3.21 | 5.03 |
| 27 | 29.28 | 3.05 | 2.63 |
| 28 | 30.31 | 2.95 | 4.08 |
| 29 | 31.42 | 2.85 | 2.50 |

### Example 4—Besylate Form A

### Preparation Method:

1. Freeform Form A (560.1 mg) was weighed into a 20 mL glass vial. Acetone (14 mL) was added to dissolve Freeform Form A solids.
2. Benzenesulfonic acid (11.0 mg) was weighed into an HPLC vial. Acetone solution of Freeform Form A (0.5 mL) was added into the vial.
3. The mixture was magnetically stirred at room temperature (RT) for 3 days.
4. The suspension was centrifuged (10000 rpm, 2 min) and solids were dried solid at RT under vacuum for 1 day.

The resulting solid was analyzed by XRPD, TGA, and DSC as described above. The results are shown in FIGS. 4A-4C. By TGA, the sample showed weight loss of 1.14% up to 150° C. DSC showed an endotherm at 165.0° C. (peak temperature).

TABLE 7
Diffraction peaks of Besylate Form A

| #Peak | 2θ[°] | d-spacing [Å] | Rel. Int. [%] |
|---|---|---|---|
| 1 | 9.27 | 9.54 | 100.00 |
| 2 | 11.14 | 7.94 | 23.51 |
| 3 | 12.32 | 7.19 | 5.63 |
| 4 | 12.78 | 6.93 | 16.69 |
| 5 | 13.51 | 6.56 | 17.75 |
| 6 | 15.43 | 5.74 | 7.86 |
| 7 | 16.68 | 5.32 | 25.82 |
| 8 | 18.56 | 4.78 | 18.09 |
| 9 | 19.57 | 4.54 | 60.58 |
| 10 | 19.83 | 4.48 | 6.75 |
| 11 | 20.32 | 4.37 | 12.90 |
| 12 | 20.72 | 4.29 | 16.56 |
| 13 | 21.24 | 4.18 | 38.86 |
| 14 | 21.66 | 4.10 | 7.49 |
| 15 | 22.39 | 3.97 | 1.61 |
| 16 | 22.94 | 3.88 | 3.14 |
| 17 | 23.29 | 3.82 | 5.63 |
| 18 | 23.82 | 3.73 | 34.04 |
| 19 | 24.21 | 3.68 | 7.58 |
| 20 | 24.74 | 3.60 | 24.61 |
| 21 | 25.67 | 3.47 | 2.89 |
| 22 | 26.18 | 3.40 | 3.97 |
| 23 | 27.97 | 3.19 | 15.95 |
| 24 | 28.39 | 3.14 | 4.79 |
| 25 | 28.67 | 3.11 | 9.89 |
| 26 | 29.35 | 3.04 | 6.68 |
| 27 | 30.64 | 2.92 | 1.08 |
| 28 | 31.28 | 2.86 | 4.63 |
| 29 | 33.03 | 2.71 | 1.42 |
| 30 | 33.71 | 2.66 | 2.51 |
| 31 | 34.03 | 2.63 | 1.49 |
| 32 | 37.04 | 2.43 | 0.97 |
| 33 | 37.57 | 2.39 | 1.83 |
| 34 | 38.26 | 2.35 | 1.03 |

### Example 5—Besylate Form B

### Preparation Method:

1. Freeform Form A (300.0 mg) and benzenesulfonic acid (164.3 mg) was weighed into a 20 mL glass vial. Acetone (6 mL) was added into the vial.
2. The mixture was magnetically stirred at room temperature (RT) for 2 days.
3. The suspension was centrifuged (10000 rpm, 2 min) and solids were dried solid at RT under vacuum for 3 days.

The resulting solid was analyzed by XRPD, TGA, and DSC as described above. The results are shown in FIGS. 5A-5C. By TGA, the sample showed weight loss of 0.84% up to 150° C. DSC showed an endotherm at 185.5° C. (peak temperature).

TABLE 8
Diffraction peaks of Besylate Form B

| #Peak | 2θ[°] | d-spacing [Å] | Rel. Int. [%] |
|---|---|---|---|
| 1 | 7.49 | 11.80 | 11.91 |
| 2 | 9.37 | 9.44 | 40.86 |
| 3 | 11.03 | 8.02 | 81.06 |
| 4 | 11.66 | 7.59 | 21.78 |
| 5 | 12.14 | 7.29 | 6.96 |
| 6 | 12.63 | 7.01 | 22.64 |
| 7 | 13.47 | 6.57 | 97.93 |
| 8 | 15.21 | 5.83 | 75.31 |
| 9 | 15.73 | 5.64 | 44.85 |
| 10 | 16.18 | 5.48 | 52.03 |
| 11 | 16.84 | 5.26 | 22.93 |
| 12 | 17.97 | 4.94 | 11.23 |
| 13 | 18.27 | 4.86 | 57.79 |
| 14 | 18.73 | 4.74 | 75.03 |
| 15 | 18.92 | 4.69 | 30.28 |
| 16 | 19.63 | 4.52 | 8.65 |
| 17 | 19.86 | 4.47 | 9.32 |
| 18 | 20.80 | 4.27 | 4.99 |
| 19 | 21.46 | 4.14 | 51.27 |
| 20 | 21.86 | 4.07 | 10.78 |
| 21 | 22.10 | 4.02 | 3.58 |
| 22 | 22.47 | 3.96 | 39.21 |
| 23 | 22.84 | 3.89 | 16.72 |
| 24 | 23.38 | 3.80 | 10.90 |
| 25 | 23.85 | 3.73 | 100.00 |
| 26 | 24.19 | 3.68 | 20.63 |
| 27 | 24.83 | 3.59 | 20.27 |
| 28 | 25.20 | 3.53 | 49.14 |
| 29 | 25.80 | 3.45 | 7.88 |
| 30 | 26.40 | 3.38 | 12.95 |
| 31 | 26.72 | 3.34 | 15.65 |
| 32 | 27.07 | 3.29 | 9.48 |
| 33 | 27.66 | 3.23 | 2.14 |
| 34 | 28.11 | 3.17 | 27.01 |
| 35 | 29.08 | 3.07 | 12.71 |
| 36 | 29.40 | 3.04 | 3.73 |
| 37 | 29.64 | 3.01 | 2.10 |
| 38 | 30.11 | 2.97 | 2.62 |
| 39 | 30.83 | 2.90 | 2.20 |
| 40 | 31.81 | 2.81 | 4.31 |
| 41 | 32.19 | 2.78 | 5.09 |
| 42 | 32.66 | 2.74 | 4.98 |
| 43 | 33.21 | 2.70 | 6.23 |
| 44 | 33.60 | 2.67 | 1.86 |
| 45 | 34.47 | 2.60 | 3.63 |
| 46 | 35.14 | 2.55 | 3.63 |
| 47 | 35.78 | 2.51 | 4.76 |
| 48 | 36.34 | 2.47 | 2.91 |
| 49 | 36.90 | 2.44 | 1.57 |
| 50 | 37.25 | 2.41 | 3.69 |
| 51 | 37.49 | 2.40 | 4.86 |
| 52 | 37.85 | 2.38 | 4.62 |
| 53 | 38.43 | 2.34 | 1.63 |
| 54 | 38.78 | 2.32 | 3.12 |
| 55 | 39.54 | 2.28 | 1.39 |

### Example 6—Oxalate Form A

### Preparation Method:

1. Freeform Form A (560.1 mg) was weighed into a 20 mL glass vial. Acetone (14 mL) was added to dissolve Freeform Form A solids.
2. Oxalic acid (6.3 mg) was weighed into an HPLC vial. Acetone solution of Freeform Form A (0.5 mL) was added into the vial.
3. The mixture was magnetically stirred at room temperature (RT) for 3 days.
4. The suspension was centrifuged (10000 rpm, 2 min) and solids were dried solid at RT under vacuum for 1 day.

The resulting solid was analyzed by XRPD, TGA, and DSC as described above. The results are shown in FIGS. 6A-6C. By TGA, the sample showed weight loss of 3.39% up to 150° C. DSC showed an endotherm at 152.4° C. (peak temperature).

TABLE 9
Diffraction peaks of Oxalate Form A

| #Peak | 2θ[°] | d-spacing [Å] | Rel. Int. [%] |
|---|---|---|---|
| 1 | 7.89 | 11.20 | 84.59 |
| 2 | 11.29 | 7.84 | 41.97 |
| 3 | 11.66 | 7.59 | 98.58 |
| 4 | 12.15 | 7.29 | 70.09 |
| 5 | 13.76 | 6.44 | 34.63 |
| 6 | 13.94 | 6.35 | 64.02 |
| 7 | 14.62 | 6.06 | 43.74 |
| 8 | 15.78 | 5.62 | 27.43 |
| 9 | 16.46 | 5.39 | 73.76 |
| 10 | 17.17 | 5.16 | 72.37 |
| 11 | 17.33 | 5.12 | 86.24 |
| 12 | 17.63 | 5.03 | 19.32 |
| 13 | 17.97 | 4.94 | 61.34 |
| 14 | 18.25 | 4.86 | 85.20 |
| 15 | 19.42 | 4.57 | 74.23 |
| 16 | 20.28 | 4.38 | 6.81 |
| 17 | 20.63 | 4.31 | 39.26 |
| 18 | 22.79 | 3.90 | 39.88 |
| 19 | 23.00 | 3.87 | 60.58 |
| 20 | 23.39 | 3.80 | 57.83 |
| 21 | 23.74 | 3.75 | 100.00 |
| 22 | 24.00 | 3.71 | 14.50 |
| 23 | 24.28 | 3.67 | 40.17 |
| 24 | 24.56 | 3.63 | 22.07 |
| 25 | 25.27 | 3.52 | 21.88 |
| 26 | 25.52 | 3.49 | 27.67 |
| 27 | 25.96 | 3.43 | 20.62 |
| 28 | 26.41 | 3.37 | 13.45 |
| 29 | 26.60 | 3.35 | 12.86 |
| 30 | 28.15 | 3.17 | 4.23 |
| 31 | 29.01 | 3.08 | 8.28 |
| 32 | 30.13 | 2.97 | 14.34 |
| 33 | 32.58 | 2.75 | 12.01 |
| 34 | 33.32 | 2.69 | 6.43 |
| 35 | 34.55 | 2.60 | 5.27 |
| 36 | 35.67 | 2.52 | 4.21 |
| 37 | 36.20 | 2.48 | 4.65 |
| 38 | 36.80 | 2.44 | 3.21 |
| 39 | 37.86 | 2.38 | 4.36 |
| 40 | 38.32 | 2.35 | 4.67 |

### Example 7—Gentisate Form A

### Preparation Method:

1. Freeform Form A (300.7 mg) and gentisic acid (160.2 mg) was weighed into a 20 mL glass vial. Ethanol (6 mL) was added into the vial.
2. The mixture was magnetically stirred at room temperature (RT) for 2 days.
3. The suspension was centrifuged (10000 rpm, 2 min) and solids were dried solid at RT under vacuum for 3 days.

The resulting solid was analyzed by XRPD, TGA, and DSC as described above. The results are shown in FIGS. 7A-7C. By TGA, the sample showed weight loss of 1.36% up to 150° C. DSC showed an endotherm at 201.0° C. (peak temperature)

TABLE 10
Diffraction peaks of Gentisate Form A

| #Peak | 2θ[°] | d-spacing [Å] | Rel. Int. [%] |
|---|---|---|---|
| 1 | 6.84 | 12.93 | 2.30 |
| 2 | 8.93 | 9.91 | 28.39 |
| 3 | 11.37 | 7.78 | 14.53 |
| 4 | 12.84 | 6.89 | 18.87 |
| 5 | 13.22 | 6.70 | 10.47 |
| 6 | 13.78 | 6.43 | 68.05 |
| 7 | 14.07 | 6.30 | 9.14 |
| 8 | 15.31 | 5.79 | 2.67 |
| 9 | 16.09 | 5.51 | 11.28 |
| 10 | 16.56 | 5.35 | 100.00 |
| 11 | 16.92 | 5.24 | 32.54 |
| 12 | 17.15 | 5.17 | 21.21 |
| 13 | 17.59 | 5.04 | 8.83 |
| 14 | 17.97 | 4.94 | 6.49 |
| 15 | 19.17 | 4.63 | 99.25 |
| 16 | 19.47 | 4.56 | 14.71 |
| 17 | 20.13 | 4.41 | 4.85 |
| 18 | 20.43 | 4.35 | 13.07 |
| 19 | 20.75 | 4.28 | 43.40 |
| 20 | 21.04 | 4.22 | 14.46 |
| 21 | 21.62 | 4.11 | 20.20 |
| 22 | 22.09 | 4.02 | 17.99 |
| 23 | 22.38 | 3.97 | 7.97 |
| 24 | 22.91 | 3.88 | 4.25 |
| 25 | 23.64 | 3.76 | 8.29 |
| 26 | 24.19 | 3.68 | 18.30 |
| 27 | 24.42 | 3.65 | 11.96 |
| 28 | 24.93 | 3.57 | 10.47 |
| 29 | 25.22 | 3.53 | 12.66 |
| 30 | 26.68 | 3.34 | 15.65 |
| 31 | 27.15 | 3.28 | 3.80 |
| 32 | 27.81 | 3.21 | 45.51 |
| 33 | 29.06 | 3.07 | 11.22 |
| 34 | 31.12 | 2.87 | 3.74 |
| 35 | 32.55 | 2.75 | 1.98 |
| 36 | 33.97 | 2.64 | 2.19 |
| 37 | 34.40 | 2.61 | 2.31 |
| 38 | 34.97 | 2.57 | 3.65 |
| 39 | 35.69 | 2.52 | 1.58 |
| 40 | 37.72 | 2.38 | 7.80 |
| 41 | 38.94 | 2.31 | 2.56 |

### Example 8 Solid State Stability

To evaluate the solid state stability of Freeform Form A, HCl Form A, Besylate Form B, and Gentisate Form A, samples were stored at 25° C./60% RH and 40° C./75% RH for 1 week. XRPD and HPLC purity was performed for stability samples to assess the physical and chemical stability.

XRPD result showed no form change for HCl Form A (FIG. 2D), Besylate Form B (FIG. 5D), and Gentisate Form A (FIG. 7D) after stored at two conditions: 25° C./60% RH/open/1 week and 40° C./75% RH/open/1 week. For Freeform Form A (FIG. 1D), amorphous was observed after stored at two conditions.

HPLC result showed that slight purity decrease was observed for HCl Form A, Besylate Form B, and Gentisate Form A and Freeform Form A after stored at 40° C./75% RH for 1 week.

TABLE 11
Solid State Stability

| | | Purity | |
|---|---|---|---|
| Sample ID | Condition | (area %) | Form change |
| Freeform | Initial | 98.15 | — |
| Form A | 25° C./60% RH/open/1 week | 98.14 | Amorphous |
| | 40° C./75% RH/open/1 week | 98.01 | Amorphous |
| HCl Form A | Initial | 97.15 | — |
| | 25° C./60% RH/open/1 week | 97.06 | No |
| | 40° C./75% RH/open/1 week | 97.00 | No |
| Besylate | Initial | 97.21 | — |
| Form B | 25° C./60% RH/open/1 week | 97.16 | No |
| | 40° C./75% RH/open/1 week | 97.12 | No |
| Gentisate | Initial | 97.22 | — |
| Form A | 25° C./60% RH/open/1 week | 97.18 | No |
| | 40° C./75% RH/open/1 week | 97.11 | No |

### Example 9 Hygroscopicity

Hygroscopicity of Freeform Form A, HCl Form A, Besylate Form B, and Gentisate Form A was evaluated by DVS.

For Besylate Form B (FIG. 5E) and Gentisate Form A (FIG. 7E), the water uptake at 25° C./80% RH was 0.32% and 0.17%, respectively. No form change was observed by XRPD after DVS test for Besylate Form B (FIG. 5F) or Gentisate Form A (FIG. 7F).

For HCl salt Form A (FIG. 2E), the water uptake at 25° C./80% RH was 0.72%. When the humidity was >80% RH, the water uptake increased (to 18.46% at 95% RH). No form change was observed by XRPD after DVS test (FIG. 2F).

For Freeform Form A (FIG. 1E) deliquesced during DVS test and the form changed to amorphous (FIG. 1F) as observed by XRPD.

| | Water uptake at 25° C./80% RH | |
|---|---|---|
| Sample ID | (wt %) | Form change |
| Freeform Form A | 1.88~5.62 | Amorphous* |
| HCl salt Form A | 0.72 | No |
| Besylate Form B | 0.32 | No |
| Gentisate Form A | 0.17 | No |

*Deliquesced during DVS test.

### Example 10 Kinetic Solubility

### Kinetic Solubility was Analyzed as Follows:

1. Add about 40 mg of samples and 4 mL of corresponding medium into a 5-mL glass vial. The loading concentration was ˜10 mg/mL (calculated by freebase).
2. Roll at 37° C. (˜25 rpm). Sample at 1, 2, 4 and 24 hrs.
3. At each time-point, sample 0.8 mL of solution or suspension into a centrifuge tube prior to centrifugation (12000 rpm, 37° C., 5 min) and filtration through 0.45 μm PTFE membrane.
4. Test pH and concentration for the filtrate. Analyze residual solids by XRPD.

### Results are Shown in the Following Table:

| | | 1 hr | | | 2 hrs | | | 4 hrs | | | 24 hrs | | |
|---|---|---|---|---|---|---|---|---|---|---|---|---|---|
| Sample | Media | S | pH | FC | S | pH | FC | S | pH | FC | S | pH | FC |
| Freeform Form A | H₂O | >9.1 | 9.4 | — | >9.3 | 9.4 | — | >9.4 | 9.4 | — | >9.3 | 9.4 | — |
| | SGF | >8.6 | 7.6 | — | >9.2 | 7.5 | — | >8.4 | 7.5 | — | >8.7 | 7.5 | — |
| | FaSSIF | >8.7 | 7.7 | — | >8.7 | 7.7 | — | >8.3 | 7.7 | — | >8.5 | 7.7 | — |
| | FeSSIF | >8.7 | 6.3 | — | >9.8 | 6.2 | — | >9.1 | 6.3 | — | >9.9 | 6.2 | — |
| HCI salt Form A | H₂O | >9.7 | 5.8 | — | >9.6 | 6.0 | — | >9.6 | 5.8 | — | >11.8* | NA | — |
| | SGF | >9.4 | 1.9 | — | >9.8 | 2.0 | — | >8.8 | 1.7 | — | >9.3 | 1.8 | — |
| | FaSSIF | >9.7 | 6.4 | — | >9.4 | 6.4 | — | >8.8 | 6.4 | — | >9.1 | 6.3 | — |
| | FeSSIF | >9.2 | 5.0 | — | >9.6 | 5.2 | — | >8.9 | 5.2 | — | >9.0 | 5.2 | — |
| Besylate Form B | H₂O | >9.3 | 5.6 | — | >9.2 | 5.8 | — | >8.9 | 5.5 | — | >9.4 | 5.8 | — |
| | SGF | >9.3 | 1.9 | — | >9.0 | 2.0 | — | >8.6 | 1.7 | — | >8.9 | 1.8 | — |
| | FaSSIF | >9.2 | 6.3 | — | >9.3 | 6.4 | — | >8.7 | 6.4 | — | >9.2 | 6.4 | — |
| | FeSSIF | >9.0 | 5.2 | — | >9.1 | 5.2 | — | >8.6 | 5.2 | — | >8.8 | 5.2 | — |
| Gentisate Form A | H₂O | 3.3 | 6.1 | No | 3.1 | 6.3 | No | 3.1 | 6.4 | No | 3.2 | 6.0 | No |
| | SGF | 6.0 | 2.7 | No | 5.9 | 2.8 | No | 5.9 | 2.7 | No | 6.0 | 2.7 | No |
| | FaSSIF | 3.9 | 6.4 | No | 3.7 | 6.5 | No | 3.8 | 6.5 | No | 4.0 | 6.4 | No |
| | FeSSIF | 4.5 | 5.1 | No | 4.2 | 5.2 | No | 4.2 | 5.2 | No | 4.5 | 5.2 | No |

S: Solubility (mg/mL), calculated by freebase.
FC: Form change.
—: Clear solution was obtained, no solid for XRPD analysis.
*: The result was higher than the loading concentration, which might be caused by evaporation of medium.

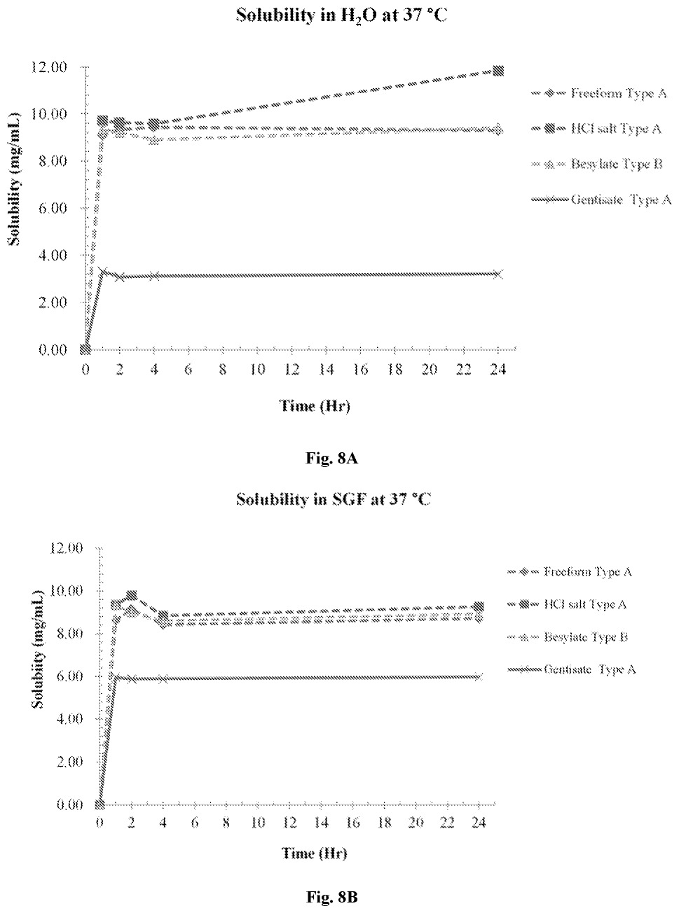

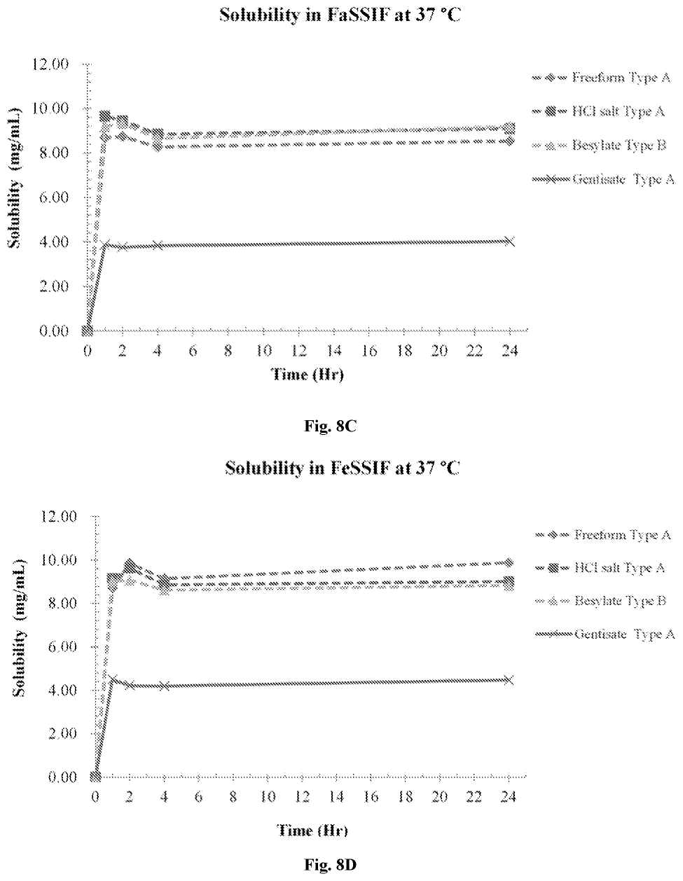

Kinetic solubility of Freeform Form A, HCl Form A, Besylate Form B, and Gentisate Form A in H₂O and bio-relevant media (SGF, FaSSIF and FeSSIF) was measured at 37° C. (FIGS. 8A-8D)

The Result Showed that:
a. For Freeform Form A, HCl salt Form A and Besylate Form B: The solubility in H₂O, SGF, FaSSIF and FeSSIF at 1, 2, 4 and 24 hrs were >8.3 mg/mL (clear solution was observed).
b. For Gentisate Form A: Lower solubility than Freeform Form A, HCl Form A and Besylate Form B was observed in all media. The highest solubility was observed in SGF. After solubility test, no form change was observed in all media (FIGS. 7G-I).

### 2. INCORPORATION BY REFERENCE

a. All of the U.S. patents and U.S. and PCT patent application publications cited herein are hereby incorporated by reference.

### 3. EQUIVALENTS

a. Those skilled in the art will recognize, or be able to ascertain using no more than routine experimentation, many equivalents to the specific embodiments of the invention described herein. Such equivalents are encompassed by the following claims.

1-65. (canceled)

66. A crystalline besylate salt of mesembrine having an X-ray powder diffraction (XRPD) pattern comprising 2-theta peaks (2 theta±0.2) 9.3°, 19.6°, 21.2°, 23.8°, and 28.0°.

67. The crystalline form of the besylate salt of mesembrine of claim 66, wherein the mesembrine is (−) mesembrine.

68. The crystalline form of the besylate salt of mesembrine of claim 67, having an X-ray powder diffraction (XRPD) pattern comprising 2-theta peaks (2 theta±0.2) 9.3°, 11.1°, 12.8°, 13.5°, 16.7°, 18.6°, 19.6°, 20.3°, 20.7°, 21.2°, 23.8°, 24.7°, and 28.0°.

69. The crystalline form of the besylate salt of mesembrine of claim 67, characterized by an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.3 | 9.5 |
| 19.6 | 4.5 |
| 21.2 | 4.2 |
| 23.8 | 3.7 |
| 24.7 | 3.6 |

70. The crystalline form of the besylate salt of mesembrine of claim 67, further characterized by a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂ |

71. The crystalline form of the besylate salt of mesembrine of claim 67, further characterized by a differential scanning calorimetry (DSC) having an endotherm at about 163° C. to about 168° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂ |

72. The crystalline form of the besylate salt of mesembrine of claim 68, further characterized by:
a. an X-ray powder diffraction (XRPD) having 2-theta peaks (2 theta±0.2) and corresponding d-spacing (angstroms±0.2):

| 2θ ± 0.2 [°] | d-spacing ± 0.2 [Å] |
|---|---|
| 9.3 | 9.5 |
| 19.6 | 4.5 |
| 21.2 | 4.2 |
| 23.8 | 3.7 |
| 24.7 | 3.6; |

b. a sample weight loss of up to about 1.5% upon heating up to 150° C. measured by TGA according to the following parameters:

| | |
|---|---|
| Parameters | TGA |
| Method | Ramp |
| Sample pan | Aluminum, open |
| Temperature | RT-350° C. |
| Heating rate | 10° C./min |
| Purge gas | N₂; and |

c. a differential scanning calorimetry (DSC) having an endotherm at about 163° C. to about 168° C. (peak temperature), measured by DSC according to the following parameters:

| | |
|---|---|
| Parameters | DSC |
| Method | Ramp |
| Sample pan | Aluminum, crimped |
| Temperature | 25° C.-230° C. |
| Heating rate | 10° C./min |
| Purge gas | N₂. |

73. The crystalline form of the besylate salt of mesembrine of claim 66, wherein the besylate salt of mesembrine is Besylate Form A.

74. A pharmaceutical composition comprising the crystalline form of the besylate salt of mesembrine of claim 66.

75. A method of treating anxiety, stress, or depression, comprising administering to a mammal in need thereof an effective amount of the pharmaceutical composition of claim 74.

76. A (−) mesembrine solid form selected from the group consisting of:
a. a solid freeform of mesembrine having an X-ray powder diffraction (XRPD) pattern comprising 2-theta peaks (2 theta±0.2) 12.9°, 13.9°, 14.9°, 19.8°, and 23.0°;
b. a crystalline HCl salt of mesembrine, having an X-ray powder diffraction (XRPD) pattern comprising 2-theta peaks (2 theta±0.2) 13.9°, 16.5°, 17.3°, 21.0°, and 27.3°;
c. a crystalline tartrate salt of mesembrine, having an X-ray powder diffraction (XRPD) pattern comprising 2-theta peaks (2 theta±0.2) 5.2°, 10.3°, 12.9°, 16.7°, and 20.7°;
d. a crystalline besylate salt of mesembrine, having an X-ray powder diffraction (XRPD) pattern comprising 2-theta peaks (2 theta±0.2) 9.3°, 19.6°, 21.2°, 23.8°, and 24.7°;
e. a crystalline besylate salt of mesembrine, having an X-ray powder diffraction (XRPD) pattern comprising 2-theta peaks (2 theta±0.2) 11.0°, 13.5°, 15.2°, 18.7°, and 23.8°;
f. a crystalline oxalate salt of mesembrine, having an X-ray powder diffraction (XRPD) pattern comprising 2-theta peaks (2 theta±0.2) 7.9°, 11.7°, 17.3°, 18.2°, and 23.7°; and
g. a crystalline gentisate salt of mesembrine, having an X-ray powder diffraction (XRPD) pattern comprising 2-theta peaks (2 theta±0.2) 13.8°, 16.6°, 19.2°, 20.8°, and 27.8°.

77. The solid form of mesembrine of claim 76, wherein the solid form is a solid freeform of mesembrine having an X-ray powder diffraction (XRPD) pattern comprising 2-theta peaks (2 theta±0.2) 12.9°, 13.9°, 14.9°, 19.8°, and 23.0°.

78. The solid form of mesembrine of claim 76, wherein the solid form is a crystalline HCl salt of mesembrine, having an X-ray powder diffraction (XRPD) pattern comprising 2-theta peaks (2 theta±0.2) 13.9°, 16.5°, 17.3°, 21.0°, and 27.3°.

79. The solid form of mesembrine of claim 76, wherein the solid form is a crystalline tartrate salt of mesembrine, having an X-ray powder diffraction (XRPD) pattern comprising 2-theta peaks (2 theta±0.2) 5.2°, 10.3°, 12.9°, 16.7°, and 20.7°.

80. The solid form of mesembrine of claim 76, wherein the solid form is a crystalline besylate of mesembrine, having an X-ray powder diffraction (XRPD) pattern comprising 2-theta peaks (2 theta±0.2) 9.3°, 19.6°, 21.2°, 23.8°, and 24.7°.

81. The solid form of mesembrine of claim 76, wherein the solid form is a crystalline besylate salt of mesembrine, having an X-ray powder diffraction (XRPD) pattern comprising 2-theta peaks (2 theta±0.2) 11.0°, 13.5°, 15.2°, 18.7°, and 23.8°.

82. The solid form of mesembrine of claim 76, wherein the solid form is a crystalline oxalate salt of mesembrine, having an X-ray powder diffraction (XRPD) pattern comprising 2-theta peaks (2 theta±0.2) 7.9°, 11.7°, 17.3°, 18.2°, and 23.7°.

83. The solid form of mesembrine of claim 76, wherein the solid form is a crystalline gentisate salt of mesembrine, having an X-ray powder diffraction (XRPD) pattern comprising 2-theta peaks (2 theta±0.2) 13.8°, 16.6°, 19.2°, 20.8°, and 27.8°.

84. A pharmaceutical composition, comprising a mesembrine solid form of claim 76; and a pharmaceutically acceptable excipient.

85. A method of treating anxiety, stress, or depression, comprising administering to a mammal in need thereof an effective amount of the pharmaceutical composition of claim 84.
